(19) United States
(12) Patent Application Publication
(10) Pub. No.: US 2026/0076797 A1
(43) Pub. Date: Mar. 19, 2026

(54) TRANSCATHETER HEART VALVES WITH IMPROVED BLOOD FLOW

(71) Applicant: Edwards Lifesciences Corporation, Irvine, CA (US)

(72) Inventors: Matthew A. Peterson, Costa Mesa, CA (US); Siddharth Vad, Irvine, CA (US); David Robert Landon, Huntington Beach, CA (US); Ram Proshad Ghosh, Anaheim, CA (US); Olivia Marie Conroy, New Rochelle, NY (US); Rani Abdullah Mahmoudi, Huntington Beach, CA (US)

(21) Appl. No.: 19/398,921

(22) Filed: Nov. 24, 2025

Related U.S. Application Data
(63) Continuation of application No. PCT/US2024/031492, filed on May 29, 2024.
(60) Provisional application No. 63/470,097, filed on May 31, 2023.
(60) Provisional application No. 63/470,729, filed on Jun. 2, 2023.

(51) Int. Cl. A61F 2/24

(52) U.S. Cl. CPC A61F 2/2418; A61F 2220/0008; A61F 2230/001; A61F 2230/0086; A61F 2250/0069

(57) ABSTRACT

A prosthetic valve includes a valve frame, an outer sealing frame, and leaflets positioned about a flow channel. Each leaflet includes two commissure ends, and a central portion positioned between the commissure ends. The leaflets are attached to form commissure regions at which commissure ends meet. The commissure regions are affixed to the valve frame, which provides openings between the commissure regions. The outer sealing frame is coupled to the valve frame such that (i) the downstream portion is spaced radially outward from the valve frame to engage and seal against the native atrioventricular valve while defining a gap between the downstream portion and the valve frame, and (ii) ventricular blood passes along radially outward facing surfaces of the leaflets and through the openings into the gap in a manner that washes out the downstream portion to reduce thrombus formation. Other implementations are also disclosed.

### CROSS-REFERENCES TO RELATED APPLICATIONS

The present application is a Continuation of International Patent Application PCT/US2024/031492 to Peterson et al., filed 29 May 2024, which published as WO 2024/249522, and which claims the benefit of (i) Provisional U.S. Patent Application 63/470,097, filed 31 May 2023, and (ii) Provisional U.S. Patent Application 63/470,729, filed 2 Jun. 2023. Each of these is incorporated herein by reference.

### FIELD OF THE INVENTION

Certain examples disclosed herein relate generally to implants, including prosthetic valves, for implantation.

### BACKGROUND

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

These replacement valves are often intended to at least partially block blood flow. However, a problem may occur upon blood stagnating within or around the replacement valve. For example, stagnation may result in formation of thrombosis or other maladies, which may result in stroke or damage to the replacement valve, among other undesired results.

Further, a problem occurs when blood flows around the valve on the outside of the prosthesis. For example, in the context of replacement heart valves, paravalvular leakage (PVL) has proven particularly challenging that in extreme cases can require reintervention to treat. An additional challenge relates to the ability of such prostheses to be secured relative to intralumenal tissue, e.g., tissue within any body lumen or cavity, in an atraumatic manner. Furthermore, in order to secure into the intralumenal tissue, another unintended consequence of such minimally invasive replacement heart valves is to secure into the heart without causing disturbances to the heart's native conduction system.

### SUMMARY OF THE INVENTION

Examples of prosthetic valves are directed to improvements in blood flow. The improved blood flow results in a reduction of thrombosis formation or other maladies of the prosthetic valve. Examples comprise collapsible or expandable prosthetic valves, configured for transcatheter delivery to an implantation site.

Examples preferably include a valve frame that emulates a surgical valve structure (e.g., a surgical ring) and a sealing frame for anchoring and sealing. This combination of features allows for more physiological flow behavior and thereby reduces the need for sustained anticoagulation therapy.

Examples of prosthetic valves may be directed to improvements in anchoring to an implantation site and/or sealing flow at an implantation site, which may comprise a native valve. Examples may be configured to conform to a shape of a native valve, which may be a non-circular shape. Examples may be directed to configurations of prosthetic valve leaflets configured to reduce fluid stagnation in a neo-sinus space.

Aspects of the disclosure include a prosthetic valve for deployment in a native mitral or tricuspid valve. The prosthetic valve comprises three prosthetic valve leaflets positioned about a flow channel of the prosthetic valve, the three leaflets being attached to form three commissure regions. The prosthetic valve comprises a valve frame for supporting the prosthetic valve leaflets, wherein the valve frame is radially expandable from a compressed configuration to an expanded configuration, the valve frame including three cantilevered support arms extending in an axial direction for supporting the three commissure regions, wherein three openings are provided between the three cantilevered support arms for reducing thrombus formation and improving leaflet coaptation. The prosthetic valve comprises an outer sealing frame positioned radially outward of the valve frame, wherein the sealing frame engages the native mitral or tricuspid valve.

Aspects of the present disclosure may include a prosthetic heart valve for deployment in a native valve. The prosthetic heart valve may include a plurality of prosthetic valve leaflets. The prosthetic heart valve may include an inner frame supporting the plurality of prosthetic valve leaflets and having a proximal portion and a distal portion. The prosthetic heart valve may include a plurality of anchors positioned at the distal portion of the inner frame and adapted to capture leaflets of the native valve. The prosthetic heart valve may include a sealing body positioned radially outward from the inner frame and having a proximal end and a distal end that is coupled to the distal portion of the inner frame such that the sealing body is adapted to move with respect to the inner frame. The prosthetic heart valve may include one or more grip features positioned on the sealing body and adapted to engage a portion of the native valve.

Aspects of the present disclosure may include a prosthetic heart valve for deployment in a native valve. The prosthetic heart valve may include a valve body. The prosthetic heart valve may include a plurality of prosthetic valve leaflets coupled to the valve body, each of the plurality of prosthetic valve leaflets including: an outflow edge, a first side commissure end, a second side commissure end positioned opposite the first side commissure end, a central portion positioned between the first side commissure end and the second side commissure end, and an inflow edge positioned opposite the outflow edge, the inflow edge including at least one indentation towards the central portion.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates a perspective view of a prosthetic valve from an outflow side of the prosthetic valve.

FIG. 2 illustrates a perspective view of the prosthetic valve of FIG. 1 from an outflow side of the prosthetic valve at a position rotated from the position shown in FIG. 1.

FIG. 3 illustrates an outflow end view of the prosthetic valve shown in FIG. 1.

FIG. 4 illustrates a side view of the prosthetic valve shown in FIG. 1.

FIG. 5 illustrates a perspective view of the prosthetic valve shown in FIG. 1 from an inflow side of the prosthetic valve.

FIG. 6 illustrates a side schematic view of the prosthetic valve shown in FIG. 1.

FIG. 7 illustrates an outflow end schematic view of the prosthetic valve shown in FIG. 1.

FIG. 8 illustrates a perspective schematic view of the prosthetic valve shown in FIG. 1 from an outflow side of the prosthetic valve.

FIG. 9 illustrates a perspective view of a support arm of the prosthetic valve shown in FIG. 1.

FIG. 10 illustrates a perspective view of a support arm strut.

FIG. 11 illustrates a perspective view of two support arm stmts.

FIG. 12 illustrates a side view of a sealing frame.

FIG. 13 illustrates a schematic view of a delivery system approaching an implantation site.

FIG. 14 illustrates a cross sectional schematic view of a delivery system approaching an implantation site.

FIG. 15 illustrates a cross sectional schematic view of a prosthetic valve deployed to a native valve.

FIG. 16 illustrates a cross sectional schematic view of a prosthetic valve deployed to a native valve.

FIG. 17 illustrates a cross sectional schematic view of a prosthetic valve deployed to a native valve.

FIG. 18 illustrates a cross sectional schematic view of a prosthetic valve deployed to a native valve.

FIG. 19 illustrates an outflow end schematic view of a prosthetic valve.

FIG. 20 illustrates a perspective schematic view of the prosthetic valve shown in FIG. 19.

FIG. 21 illustrates an inflow perspective view of frames of a prosthetic valve.

FIG. 22 illustrates a side view of the frames shown in FIG. 21.

FIG. 23 illustrates a side view of a frame shown in FIG. 21.

FIG. 24 illustrates a side view of a frame shown in FIG. 21.

FIG. 25 illustrates an outflow perspective view of the frames shown in FIG. 21.

FIG. 26 illustrates a side cross sectional schematic view of a prosthetic valve.

FIG. 27 illustrates a cross sectional schematic view of a delivery system approaching an implantation site.

FIG. 28 illustrates a cross sectional schematic view of the prosthetic valve shown in FIG. 26 deployed to a native valve.

FIG. 29 illustrates a perspective view of a prosthetic valve.

FIG. 30 illustrates a top perspective view of the prosthetic valve shown in FIG. 29.

FIG. 31 illustrates a cross sectional schematic view of the prosthetic valve shown in FIG. 29.

FIG. 32 illustrates a cross sectional schematic view of the prosthetic valve shown in FIG. 29.

FIG. 33 illustrates a cross sectional schematic view of the prosthetic valve shown in FIG. 29.

FIG. 34 illustrates a perspective view of a frame for a prosthetic valve.

FIG. 35 illustrates a detail perspective view of an anchor positioned relative to a frame of a prosthetic valve.

FIG. 36 illustrates a detail side view of an anchor positioned relative to a frame of a prosthetic valve.

FIG. 37 illustrates a schematic cross sectional side view of an anchor pressing a native valve leaflet against a frame.

FIG. 38 illustrates a schematic cross sectional side view of a grip feature engaging a native valve leaflet.

FIG. 39 illustrates a schematic cross sectional side view of a grip feature engaging a native valve leaflet.

FIG. 40 illustrates a schematic view of a delivery system approaching an implantation site.

FIG. 41 illustrates a side schematic view of the prosthetic valve shown in FIG. 29 deployed to an implantation site.

FIG. 42 illustrates a perspective view of a prosthetic valve.

FIG. 43 illustrates a cross sectional schematic view of the prosthetic valve shown in FIG. 42.

FIG. 44 illustrates a cross sectional schematic view of a prosthetic valve.

FIG. 45 illustrates a cross sectional schematic view of a prosthetic valve.

FIG. 46 illustrates a cross sectional schematic view of a prosthetic valve.

FIG. 47 illustrates a flat view of a prosthetic valve leaflet.

FIG. 48 illustrates a perspective view of three prosthetic valve leaflets.

FIG. 49 illustrates a cross sectional schematic view of a prosthetic valve.

FIG. 50 illustrates a cross sectional schematic view of a portion of a prosthetic valve.

### DETAILED DESCRIPTION OF EMBODIMENTS

FIG. 1 illustrates a perspective view of a prosthetic valve 10 in the form of a replacement heart valve. The prosthetic valve 10 is configured to be deployed within a portion of a patient's body. The prosthetic valve 10, for example, is configured to be deployed to an annulus of a native valve, which may comprise a native mitral valve or a native tricuspid valve. In examples other implantation locations can be utilized such as within an aortic or pulmonary valve, or in other valves or locations within a patient's body as desired.

The prosthetic valve 10 includes a proximal end 12 (marked in FIG. 4) and a distal end 14, and a length therebetween. The proximal end 12 comprises an inflow end of the prosthetic valve 10, and the distal end 14 comprises an outflow end of the prosthetic valve 10. The prosthetic valve 10 includes a plurality of prosthetic valve leaflets 16 (e.g., three prosthetic valve leaflets) configured to surround a flow channel 13 for controlling flow through the valve 10. The prosthetic valve leaflets 16 are configured to move between opened and closed states to mimic and replace the operation of native valve leaflets.

The prosthetic valve leaflets 16 have a plurality of commissures. For example, as shown in FIG. 1, three commissures 26a, b, c or commissure regions between the respective prosthetic valve leaflets 16a, b, c may exist. The leaflets 16 are attached to form three commissures 26a, b, c, or commissure regions. The commissure 26a is positioned between the respective prosthetic valve leaflets 16a, b. The commissure 26b is positioned between the respective prosthetic valve leaflets 16b, c. The commissure 26c is positioned between the respective prosthetic valve leaflets 16c, a. The commissures 26a, b, c can be equally spaced from each other or other spacings can be utilized in examples. A lesser number of commissures can be utilized in examples in which a fewer number of prosthetic valve leaflets are utilized. For example, two commissures may exist for two prosthetic valve leaflets. A greater number of commissures can be utilized for a greater number of prosthetic valve leaflets.

In examples, the prosthetic valve 10 includes a frame 18. The frame 18 can comprise a dual frame or multi-component frame in examples. The frame 18, for example, includes a valve frame 20 and a sealing frame 22 in examples. Other configurations can be utilized in examples.

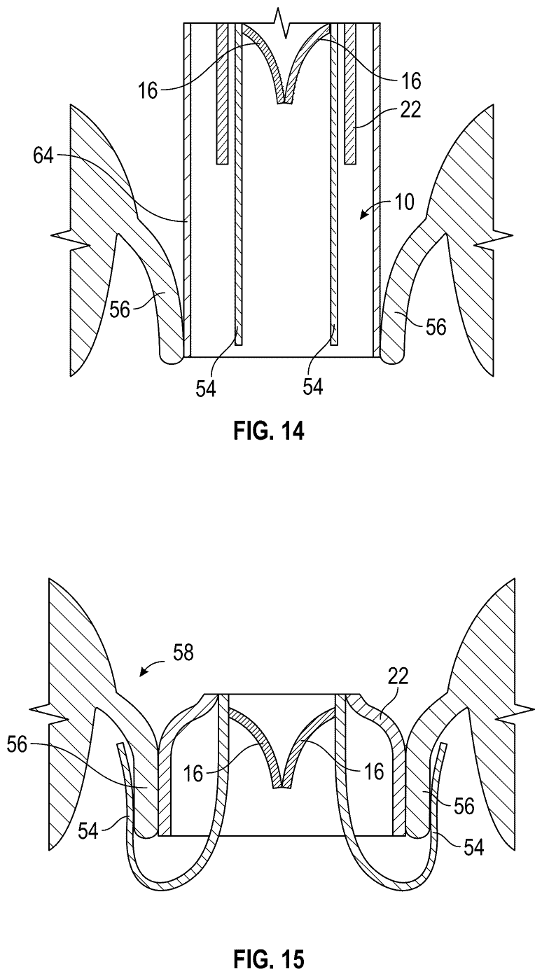

The valve frame 20 is configured to support the plurality of prosthetic valve leaflets 16. The valve frame 20 is radially expandable from a compressed configuration to an expanded configuration. The valve frame 20 is compressible from the expanded configuration radially inward to the compressed configuration. The expanded configuration is shown in FIG. 1, yet a compressed configuration (represented in FIG. 14) can be utilized. The compressed configuration is for transcatheter delivery of the prosthetic valve 10 to an implantation site.

The valve frame 20 includes one or more support arms in examples. The support arms can include a first support arm 24a that extends axially and is configured to support at least two of the plurality of prosthetic valve leaflets 16 (prosthetic valve leaflets 16a, b) at a first commissure 26a or commissure region. The support arms can include a second support arm 24b that extends axially and is configured to support at least two of the plurality of prosthetic valve leaflets 16 (prosthetic valve leaflets 16b, c) at a second commissure 26b or commissure region. In examples, a third support arm 24c extends axially and is configured to support at least two of the plurality of prosthetic valve leaflets 16 (prosthetic valve leaflets 16c, a) at a third commissure 26c or commissure region. The support arms 24a, b, c may comprise cantilevered support arms in examples. The support arms 24a, b, c extend in an axial direction.

In examples, each of the support arms 24a, b, c, are spaced circumferentially equidistant from each other. Other spacings can be utilized in examples.

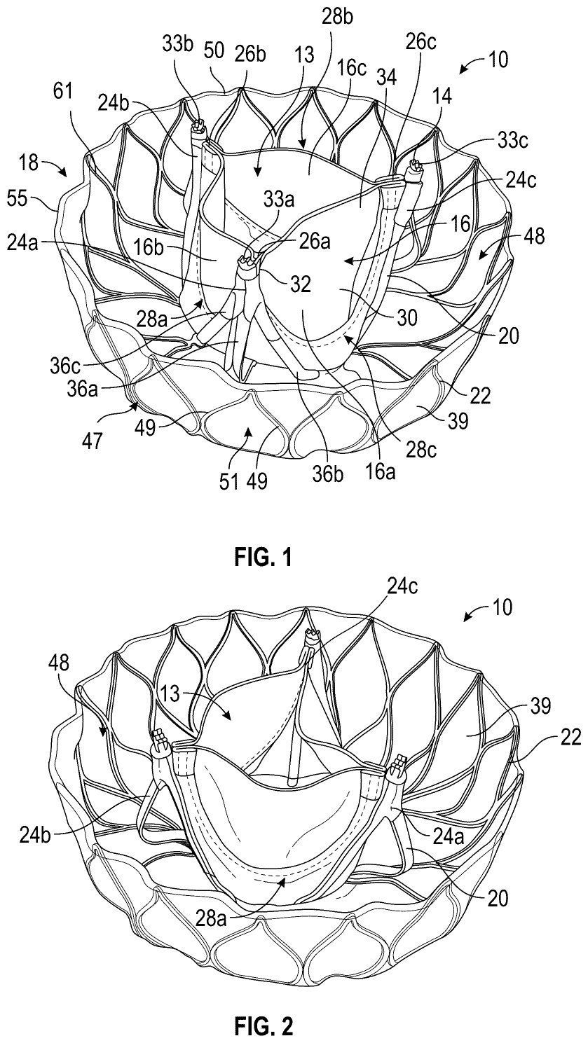

An opening 28a is positioned circumferentially between the first support arm 24a and the second support arm 24b and is configured to allow for blood flow therethrough. The opening 28a, for example, comprises a space between the first support arm 24a and the second support arm 24b that can span from the first support arm 24a to the second support arm 24b. The opening 28a comprises a flow channel for blood flow. The opening 28a can have a “U” shape in examples (as shown in FIGS. 1 and 2), or can have other configurations as desired.

An opening 28b is positioned between the support arms 24b, c. An opening 28c is positioned between the support arms 24c, a. The openings 28b, c can be configured similarly as the opening 28a in examples. The opening 28b can span from the second support arm 24b to the third support arm 24c. The opening 28c can span from the third support arm 24c to the first support arm 24a. The three openings 28a, b, c are provided between the support arms 24a, b, c. The three openings 28a, b, c reduce thrombus formation and improve leaflet coaptation. The openings 28a, b, c are substantially unobstructed to allow for improved blood flow therethrough.

Each prosthetic valve leaflet 16a, b, c includes a central portion 30 positioned between a first commissure end portion 32 and a second commissure end portion 34. The first commissure end portion 32 is positioned at a respective commissure of adjacent prosthetic valve leaflets. The second commissure end portion 34 is positioned at a respective opposite commissure of adjacent prosthetic valve leaflets.

In examples, a respective opening 28a is circumferentially aligned with the central portion 30 of the prosthetic valve leaflet 16b. Such a position allows for blood flow from a radially outward facing surface of the prosthetic valve leaflet through a respective opening (e.g., opening 28a). Washout and leaflet coaptation is accordingly improved.

Each support arm 24a, b, c can extend axially in an outflow direction to a respective free end 33a, b, c of the support arm 24a, b, c. The free end 33a, b, c can comprise the terminus of the respective support arm 24a, b, c. The ends 33a, b, c of the support arms 24a, b, c can be free and not directly connected to each other in examples. In examples, connections between the ends 33a, b, c are utilized.

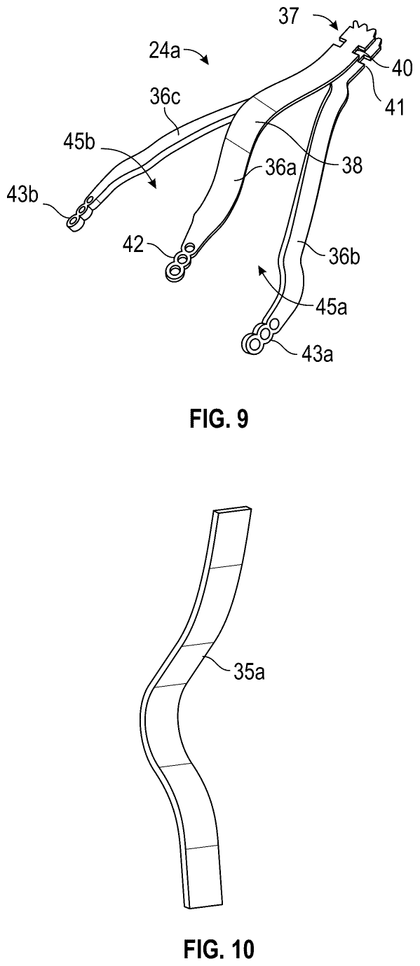

Each support arm 24a, b, c comprises one or more support arm struts in examples. For example, the support arm 24a can comprise three support arm struts 36a, b, c. The support arm strut 36a comprises a central strut that extends axially. The support arm strut 36a is positioned circumferentially between adjacent support arm struts 36b, c. A perspective view of the support arm 24a is shown in FIG. 9. The support arm strut 36a comprises an outer curved strut for resisting inward bending of the support arm 24a. The support arm strut 36a is shown with a radially outward curve portion 38. A distal end 37 of the support arm strut 36a includes a coupler 40 for coupling with the commissure 26a of the prosthetic valve leaflets 16a, b. A proximal end of the support arm strut 36a includes a coupler 42 for coupling with a portion of a delivery system for the prosthetic valve. The coupler 42 is configured for coupling with a portion of the sealing frame 22 in examples.

The curve portion 38 of the support arm strut 36a is curved to resist loads (e.g., systolic loads) on the prosthetic valve 10 and valve frame 20 applied at the commissures of the prosthetic valve leaflets 16. The curvature of the curve portion 38 resists loads on the prosthetic valve 10 and valve frame 20 that pull on the cantilevered support arm 24a at the commissures. The curve portion 38 comprises a bent portion. The shape of the curved portion or bent portion is adjustable (e.g., enhanced or reduced) as desired to vary the resistance to the load. By providing a radially outward curvature or bending, the moment of inertia of the support arm 24a can be set to resist bending radially inward. In examples, the resulting support arms 24a, b, c may be flexible in a radial direction.

The support arm strut 36a resists forces applied to the prosthetic valve 10 and valve frame 20 via its connection to the sealing frame 22. The proximal end of the support arm strut 36a comprises an inflow support semitrue that holds the inflow of the prosthetic valve 10 circular. The coupler 42 at the prosthetic valve, for example, couples to a coupler 59 at a proximal end 46 of the scaling frame 22 (as marked in FIGS. 4 and 12) to form an inflow support structure that holds the inflow of the prosthetic valve 10 circular. The support arm strut 36a extends axially, and is circumferentially aligned with a respective commissure or commissure region of the prosthetic valve leaflets.

The support arm struts 36b, c are angled towards the central support arm strut 36a. The support arm struts 36b, c comprise a unitary body for coupling to the distal end 37 of the support arm strut 36a or another configuration can be utilized in examples. The support arm struts 36b, c angle towards the central support arm strut 36a in a direction towards the distal end of the central support arm strut 36a.

The support arms struts 36b, c include a coupler 41 at a distal end for coupling with the commissure 26a of the prosthetic valve leaflets 16a, b in a similar manner as the coupler 40. The support arm struts 36b, c include respective couplers 43a, b at a proximal end for coupling with a portion of a delivery system or with a portion of the sealing frame 22 in examples.

The support arm struts 36b, c are angled towards the central support arm strut 36a to form spaces 45a, b spaced circumferentially from the central support aim strut 36a. The spaces 45a, b have triangular or wedge shapes in examples. The spaces 45a, b allow the support arm struts 36b, c to collapse towards the central support arm strut 36a in a collapsed configuration. Such a feature allows the valve frame 20 to move towards a collapsed configuration. The support arm struts 36b, c can deflect outward away from the central support arm strut 36a upon the valve frame 20 moving towards the expanded configuration.

The support arm struts 36a, b, c can be made of a self-expanding material in examples. The self-expanding material can comprise nitinol or other forms of self-expanding material. The support arm struts 36a, b, c comprise multiple thin layers of self-expanding material (e.g., nitinol) layered upon each other as shown in FIG. 9. Other configurations can be utilized in examples.

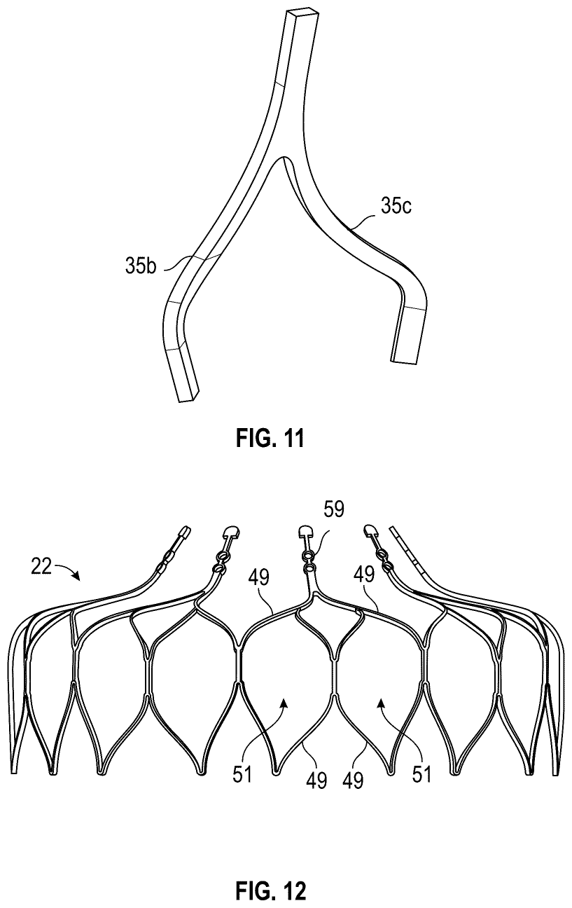

FIG. 10 illustrates an isolated view of a central support arm strut 35a that can be utilized in a similar manner as the support arm strut 36a. FIG. 11 illustrates an isolated view of angled support arm struts 35b, c that can be utilized in a similar manner as the support arm struts 36b, c. The support arm struts 36b, c have a “Y” configuration or other forked configuration as desired in examples.

In examples, other configurations of support arms can be utilized. The support arms 24a, b, c can each be configured similarly as each other or can have other configurations in examples.

The support arms 24a, b, c can be separated circumferentially from each other and not directly connected to each other in examples. Direct connections can be utilized in examples.

Referring to FIG. 1, the sealing frame 22 is configured to seal with and engage a native valve. The sealing frame 22 comprises an outer sealing frame 22 surrounding the valve frame 20 and positioned radially outward of the valve frame 20. The sealing frame 22 is configured to contact with a portion of a native valve (e.g., native valve leaflets or a native valve annulus) to seal with the portion of the native valve. In examples, a sealing skirt 39 is positioned on the sealing frame 22. The sealing frame 22 can comprise a portion of a sealing body for sealing with the portion of the native valve. The sealing body can comprise the combination of the sealing frame 22 and the sealing skirt 39. Other configurations can be utilized in examples. An outer surface 47 of the sealing body can contact the portion of the native valve to seal with the native valve. In examples, the sealing skirt 39 may comprise a fabric sealing skirt. The sealing skirt 39 may be positioned over a substantial portion of the sealing frame 22.

The sealing frame 22 is expandable from a compressed configuration radially outward to an expanded configuration. The sealing frame 22 is compressible from the expanded configuration radially inward to the compressed configuration. The sealing frame 22, for example, comprises a lattice structure of struts 49 and openings 51 or a strut lattice that comprises a compressible and expandable structure. The lattice structure is configured such that a length of the sealing frame 22 increases upon the diameter of the sealing frame 22 decreasing upon compression. The length of the sealing frame 22 decreases with the diameter of the sealing frame 22 increasing upon expansion to an expanded configuration. FIG. 12 illustrates an isolated side view of the sealing frame 22.

Referring to FIG. 4, the sealing frame 22 includes a proximal portion 53 (or inflow portion), a distal portion 55 (or outflow portion), and a curved portion 57 positioned between the proximal portion 53 and the distal portion 55. The proximal portion 53 protrudes radially outward from a proximal end 46 (or inflow end or inflow portion) of the sealing frame 22. The sealing frame 22 protrudes radially outward from the valve frame 20. The distal portion 55 extends in a distal direction. The curved portion 57 curves to angle the distal portion 55 relative to the proximal portion 53. The proximal portion 53 of the scaling frame 22 couples to a proximal portion of the valve frame 20.

The proximal end 46 of the sealing frame 22 includes one or more couplers 59 for coupling with the couplers 42, 43a, b of the support arms as shown in FIG. 9. Such coupling forms the connection between the sealing frame 22 and the valve frame 20. Other forms of connection can be utilized in examples. The couplers 59 can be utilized for coupling with a portion of a delivery system in examples. The couplers 59, for example, can comprise eyelets or other forms of couplers for connection with sutures or other connecting bodies of the delivery system. Other forms of connection can be utilized in examples.

The couplers 59 are positioned at proximal tips of the struts 49 of the sealing frame 22. A respective coupler 59 couples to a respective one of the couplers 42, 43a, b of the support arms. For example, nine support arm struts are utilized in the example shown in FIG. 1. Nine corresponding couplers 59 can be utilized (as represented in FIG. 4 for example). The couplers 59 are circumferentially spaced from each other, with adjacent groups of couplers 59 (e.g., groups of three) utilized for a respective support arm 24a, b, c.

Referring to FIG. 1, the sealing frame 22 surrounds the valve frame 20. The sealing frame 22, for example, surrounds the first support arm 24a and the second support arm 24b. The sealing frame 22 can surround the third support arm 24c in an example in which the third support arm 24c is utilized. In examples, an outlet end of the sealing frame 22 may be positioned further downstream than an outlet end of the valve frame 20. In examples, an outlet end of the valve frame 20 may be positioned further downstream than an outlet end of the sealing frame 22.

The sealing frame 22 is spaced from the valve frame 20 with a gap 48. The gap 48 forms a ring extending about the valve frame 20 and having an annular shape. The gap 48 comprises a spacing from the valve frame 20 that allows for blood flow therethrough. An interior surface 61 of the sealing frame 22 faces towards the gap 48. The outlet end of the valve frame 20 is radially spaced apart from the outlet end of the sealing frame 22 for providing the gap 48 therebetween. The outlet end of the valve frame 20 does not contact an outlet end of the sealing frame 22 and a clearance is provided therebetween.

The distal portion 55 or outlet portion of the sealing frame 22 is spaced from the valve frame 20 to form an opening 50 for an interior of the sealing frame 22. The interior of the sealing frame 22 comprises the gap 48 or annular ring extending about the valve frame 20. As such, blood can flow through the opening 50 and within the gap 48. The sealing frame 22 has a concave shape facing a ventricle when deployed for funneling blood toward the valve frame 20 and the prosthetic valve leaflets 16.

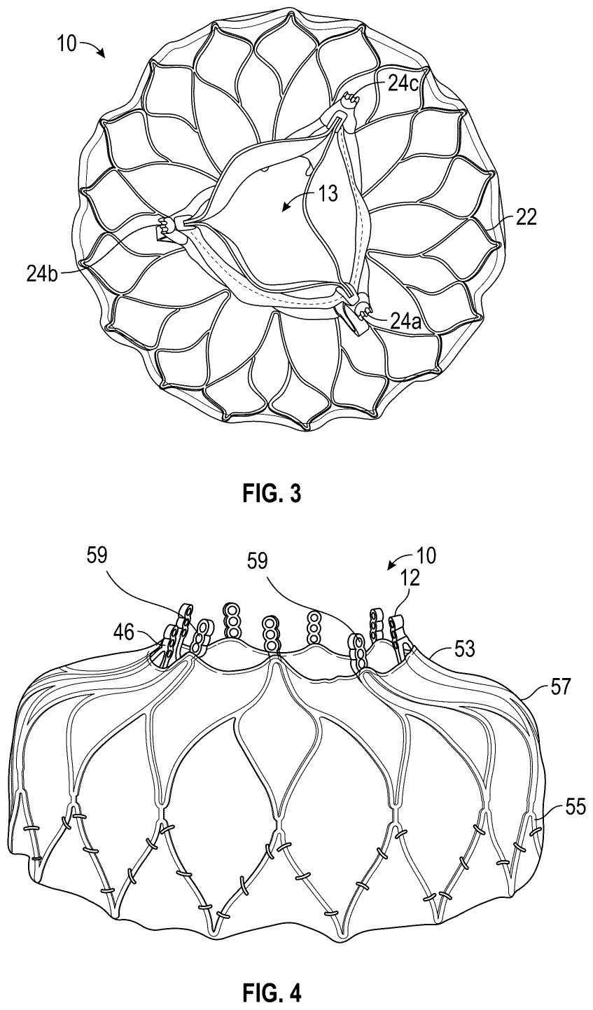

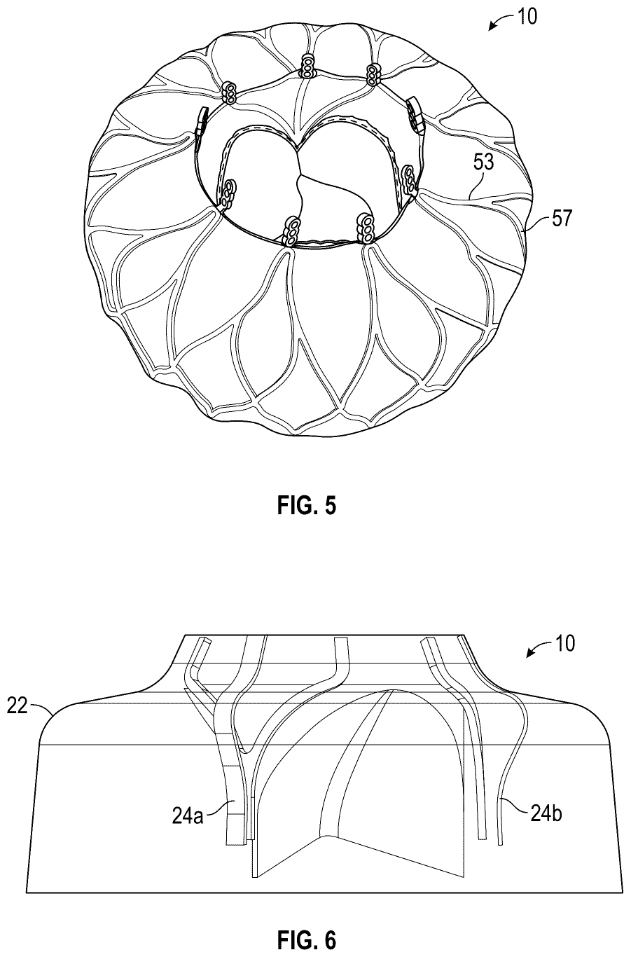

FIG. 2 illustrates a perspective view of the prosthetic valve 10 rotated from the position shown in FIG. 1. FIG. 3 illustrates a distal end view or outflow end view of the prosthetic valve 10. FIG. 4 illustrates a side view of the prosthetic valve 10. FIG. 5 illustrates a proximal end view or inflow end view of the prosthetic valve 10.

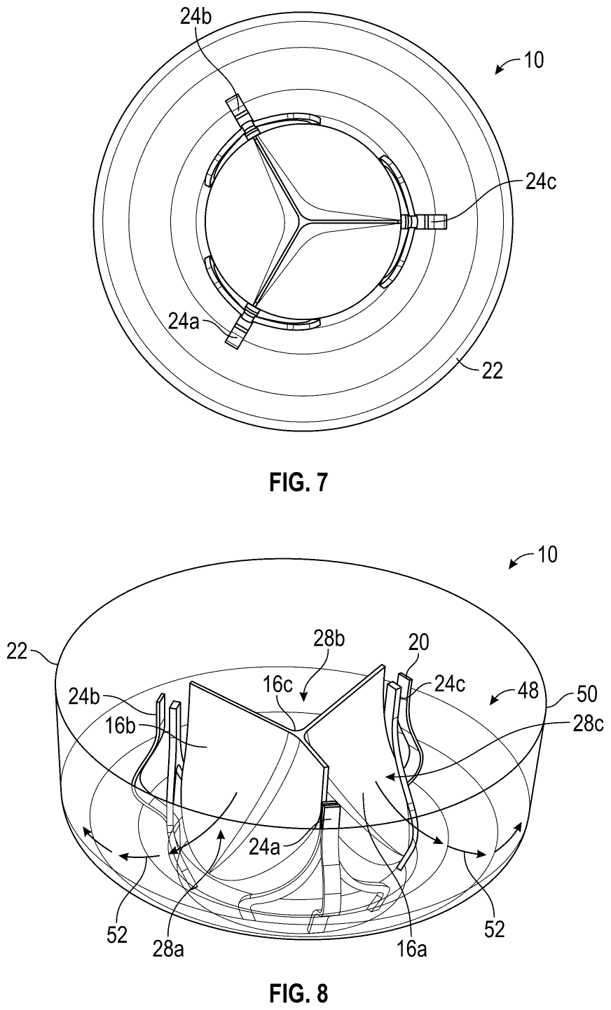

FIG. 6 illustrates a schematic representation of the prosthetic valve 10. FIG. 7 illustrates a schematic distal end view or outflow end view of the prosthetic valve 10.

Referring to FIG. 8, the openings 28a, b, c between adjacent support arms 24a, b, c allow for blood flow to pass therethrough, thereby reducing the prevalence of stagnated blood flow radially exterior of the prosthetic valve leaflets 16a, b, c. As represented with the flow arrows 52 in FIG. 8, the blood passes along the radially outward facing surfaces of the prosthetic valve leaflets 16a, b, c through the respective openings 28a, b, c. The openings 28a, b, c provide flow channels for blood flow. The blood flows through and exits the gap 48 positioned between the valve frame 20 and the sealing frame 22. The sealing frame 22 preferably has a smooth inner surface that redirects the flow and encourages complete washout when the prosthetic valve leaflets are in the closed position.

A reduced prevalence of stagnated blood flow can reduce the formation of thrombosis or other maladies radially outward of the leaflets 16 or within the gap 48 of the sealing frame 22. Improved hemodynamics result. Further, the prosthetic valve 10 is collapsible or expandable to allow for transcatheter delivery.

The valve frame 20 emulates a surgical valve structure (e.g., a surgical ring). The sealing frame 22 provides for anchoring and sealing. This combination of features allows for more physiological flow behavior and thereby reduces the need for sustained anticoagulation therapy. The low thrombogenicity benefit of a bioprosthetic surgical valve is emulated. The prosthetic valve leaflets 16 are suspended in free-space (rather than covered by a cylindrical structure). This type of structure reduces the possibility of thrombus formation due to the open flow channels between the support arms 24a, b, c. The shape of the sealing frame 22 reduces the prevalence of turbulence or clotting and allows for washout upon opening and closing of the prosthetic valve leaflets.

In examples, one or more anchors 54 (marked in FIG. 15) are utilized to anchor the prosthetic valve 10 to an implantation site. For example, referring to FIG. 15, the anchors 54 comprise curved elongate anchors or hooks configured to hook about a native valve leaflet 56 to be positioned radially outward of the native valve leaflet 56. The anchors are shaped to extend over a distal tip of a native valve leaflet 56 of the native valve 58. The anchors 54 can extend from the valve frame 20 in examples. For example, the anchors 54 can extend from a respective one of the support arms 24a, b, c in examples. Anchors can extend from other portions of a prosthetic valve 10 in examples. The anchors 54 are adapted to capture native leaflets between the anchors 54 and the sealing frame 22. The anchors 54 are adapted to bend during deployment from an elongated configuration to a hooked, curved, or bent configuration as shown in FIG. 15 for example.

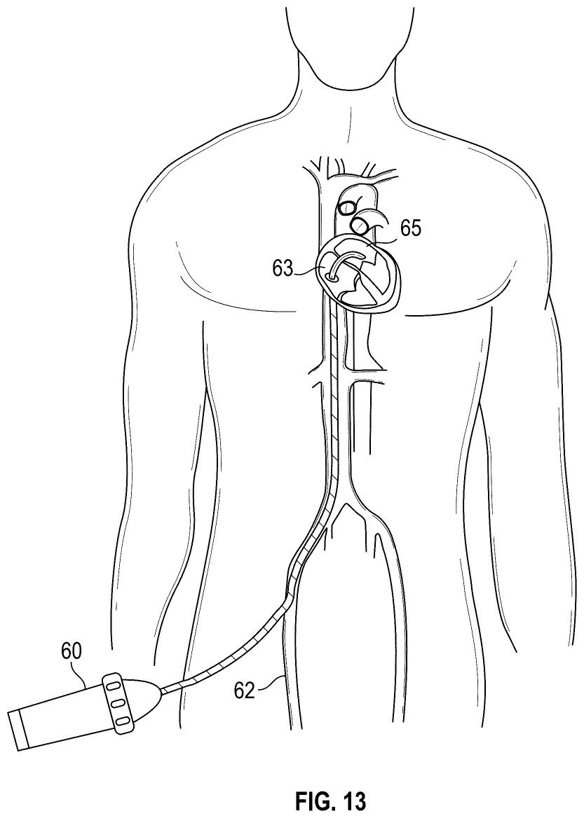

FIGS. 13-15 illustrate an exemplary deployment procedure of the prosthetic valve 10. Referring to FIG. 13, a delivery apparatus 60 can approach an implantation site via passage through the patient's vasculature. Transcatheter deployment can be utilized in examples. The delivery apparatus 60 can be passed transvenous through the femoral vein 62 or through another delivery approach in examples.

The delivery apparatus 60 can approach the implantation site transvenous. The delivery apparatus 60, for example, can approach the right atrium 63 for deployment to the tricuspid valve. The delivery apparatus 60 can pass transseptal to the left atrium 65 for deployment to the mitral valve in examples. FIG. 13 illustrates an advancement to the mitral valve, yet advancement to the tricuspid valve can be utilized. Other approaches can be utilized for other implantation sites in examples.

Referring to FIG. 14, the prosthetic valve 10 is held in a compressed configuration within the delivery apparatus 60 in examples. For example, a capsule 64 or other form of retention body can retain the prosthetic valve 10 in a compressed configuration. The prosthetic valve 10 can comprise a self-expanding prosthetic valve 10 that expands upon release by the retention body (such as the capsule 64). The frame 18 of the prosthetic valve 10 (including the valve frame 20 and sealing frame 22) is self-expanding. The frame 18 can be made of a shape memory material that produces the self-expansion. The shape memory material can be Nitinol or another form of shape memory material in examples.

In examples, the prosthetic valve 10 can be configured to be delivered with an inflatable body such as a balloon or other forms of deployment. The inflatable body is positioned within an interior of the prosthetic valve 10 and inflated to expand the prosthetic valve 10 to the expanded configuration.

FIG. 14 illustrates the prosthetic valve 10 in a compressed and elongated configuration, with the anchors 54 and sealing frame 22 elongated and having a reduced diameter from the deployed configuration. The capsule 64 can retract to release the prosthetic valve 10 from the compressed configuration, or other forms of deployment can be utilized in examples.

FIG. 15 illustrates the prosthetic valve 10 having been deployed. The anchors 54 hook over the native valve leaflets 56. Blood flow is allowed through the respective openings 28a, b, c as represented in FIG. 8 for example. Improved hemodynamics result.

Other forms of prosthetic valves can be utilized in examples. Other forms of anchoring can be utilized in examples.

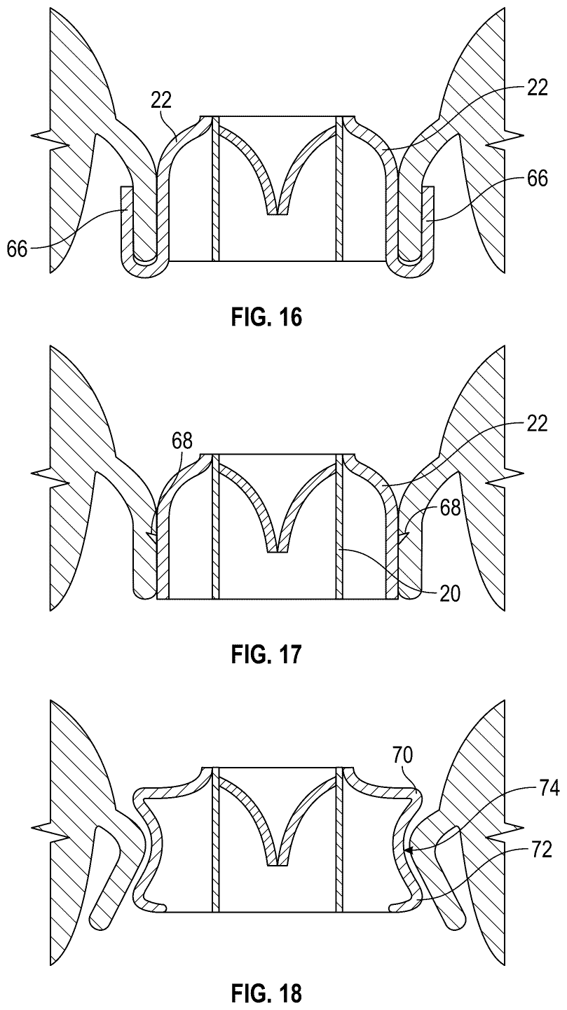

FIG. 16, for example, illustrates a variation of the prosthetic valve 10 in which anchors 66 extend from the sealing frame 22. The valve frame 20 lacks anchors directly coupled to the valve frame 20 and accordingly can have an appearance as shown in FIG. 1, for example.

FIG. 17 illustrates a variation in which one or more anchors 68 comprise barbs that extend radially outward from the sealing frame 22. The anchors 68 are positioned on the sealing frame 22 (e.g., an outer surface of the sealing frame 22) or can be positioned on the valve frame 20 and can extend radially outward from the valve frame 20 and through the sealing frame 22. The anchors 68 enhance anchoring with the native valve.

FIG. 18 illustrates a variation in which the sealing frame 22 forms one or more flanges 70, 72 for anchoring the prosthetic valve to the native valve. A proximal or atrial flange 70 can be utilized and a distal or ventricular flange 72 can be utilized. A recess 74 is formed between the proximal or atrial flange 70 and the distal or ventricular flange 72 for retaining a portion of the native valve for anchoring. The sealing frame 22 can have an hourglass shape or another shape in examples.

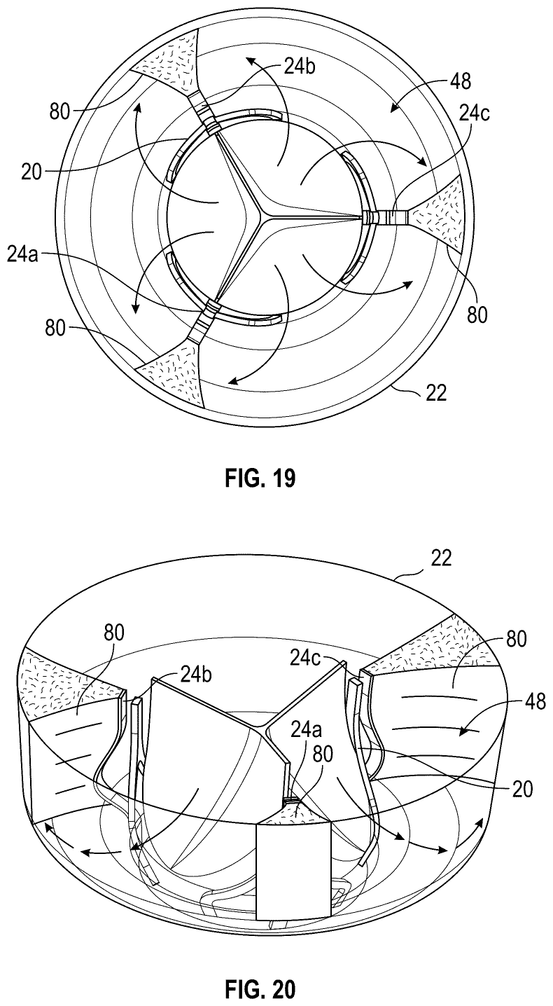

Other variations in the prosthetic valve 10 can be provided. FIG. 19, for example, illustrates a variation in which one or more flow barriers 80 are positioned within the gap 48. The flow barriers 80 are circumferentially spaced from each other and are positioned radially outward of a respective one of the support arms 24a, b, c. The flow barriers 80 extend from a respective one of the support arms 24a, b, c radially outward to the sealing frame 22. In examples, the flow barriers 80 extend radially outward from a central support arm strut 36a (as marked in FIG. 9 for example). The flow barriers 80 couple to the central support arm strut 36a and extend radially outward to the sealing frame 22. The flow barriers 80 can cover the curve portion 38 of the support arm strut 36a in examples, or other configurations can be utilized. Three flow barriers 80 may be provided although a lesser or greater number may be utilized as desired.

The flow barriers 80 are positioned to reduce the possibility of blood stagnating radially outward of the respective one of the support arms 24a, b, c. The portion of the gap 48 forming a partial ring about the valve frame 20 allows for blood flow therethrough between the flow barriers 80. Three discrete regions within the sealing frame 22 for improved washout are provided. The flow barriers 80 can have a wedge shape or tapered shape as represented in FIGS. 19 and 20. The wedge shape or tapered shape increases in width in a radially outward direction. Other shapes can be utilized in examples as desired.

The features of FIGS. 1-20 may be utilized solely or in combination with any other example herein.

Various other configurations of prosthetic valves can be utilized in examples.

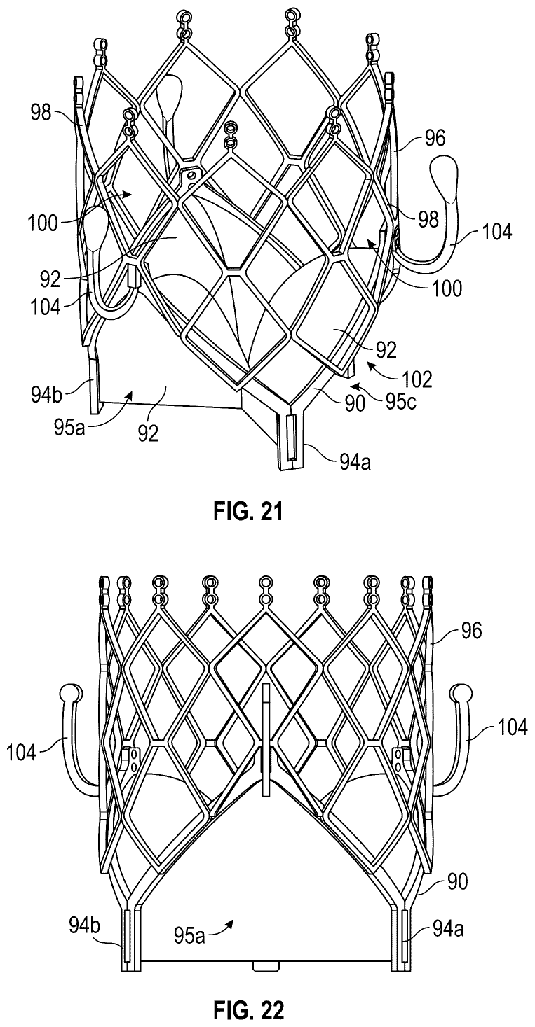

FIG. 21, for example, illustrates a configuration of frames for a prosthetic valve in which the valve frame 90 retains the prosthetic valve leaflets 92 sub annular'. The valve frame 90 can be configured similarly as the valve frame 20 and includes a plurality of support arms 94a, b, c each extending axially. The support arms 94a, b, c are positioned at the commissures or commissure regions of adjacent prosthetic valve leaflets 92. Openings 95a, b, c are positioned between respective adjacent support arms 94a, b, c. The support arms 94a, b, c may comprise cantilevered support arms in examples. The support arms 94a, b, c extend in an axial direction for supporting the commissure regions of the adjacent prosthetic valve leaflets 92. The support arms 94a, b, c support the commissure regions of the prosthetic valve leaflets 92. The openings 95a, b, c are positioned between respective adjacent support arms 94a, b, c for reducing thrombus formation and improving leaflet coaptation. The openings 95a, b, c are substantially unobstructed to allow for improved blood flow therethrough.

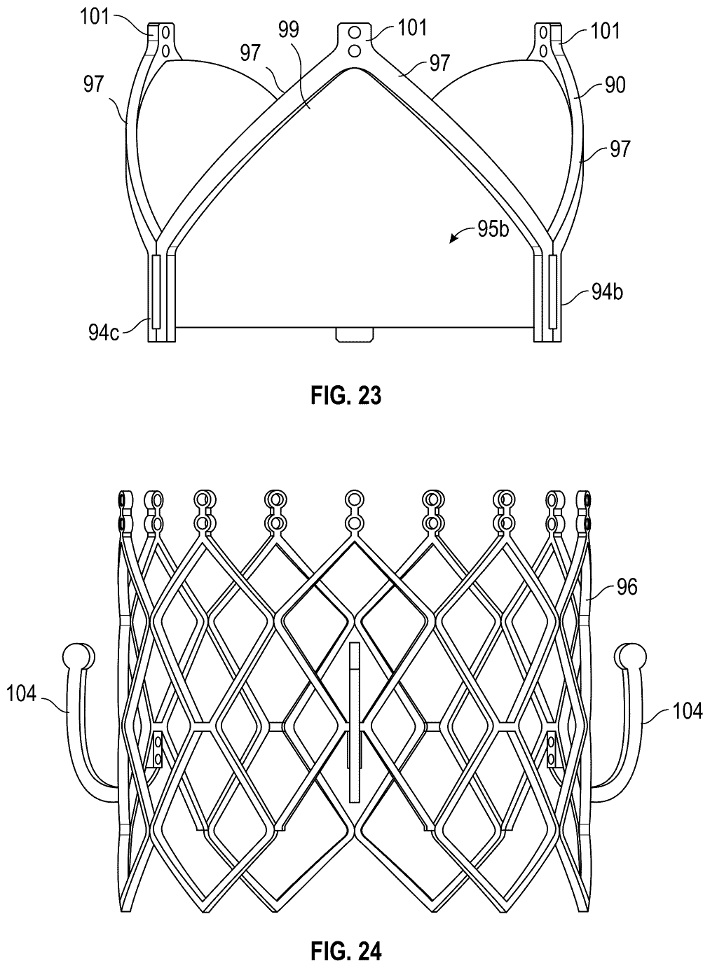

FIG. 23 illustrates a side view of the valve frame 90 at an opposite side of the valve frame 90 than shown in FIG. 21. The support arms 94a, b, c are connected with connecting struts 97 that extend along an inflow edge 99 of the respective prosthetic valve leaflets 92. The connecting struts 97 are angled to form a “Y” shape or a forked shape between adjacent of the support arms 94a, b, c. The connecting struts 97 extend to a coupler 101 at a central portion of the openings 95a, b, c of the valve frame 90 that can be utilized to couple with an anchoring frame 96.

In examples, an anchoring frame 96 comprises a lattice structure of struts 98 or strut lattice and openings 100. The anchoring frame 96 has a cylindrical shape as shown in FIG. 21 or can have another shape as desired. The anchoring frame 96 and the valve frame 90 can each be configured to be in a compressed configuration or an expanded configuration. Transcatheter delivery can be utilized.

The valve frame 90 couples to a distal end portion 102 of the anchoring frame 96 in examples. The anchoring frame 96 is positioned along an inlet end of the prosthetic valve and at an inlet portion or inlet end of the valve frame 90. The anchoring frame 96 can be modularly coupled to the valve frame 90 in examples. The anchoring frame 96 can be detached from the valve frame 90 as desired. As such, various other configurations of valve frames and anchoring frames can be interchangeably utilized as desired. Various combinations of valve frames and anchoring frames can be utilized to address a size of an implantation site or the type of implantation site (e.g., mitral implantation versus tricuspid). A different size of valve frame 90 can be modularly coupled to the anchoring frame 96 as desired. A different type of valve frame 90 (e.g., two prosthetic valve leaflets versus three prosthetic valve leaflets) can be modularly utilized. The couplers 101 shown in FIG. 23, for example, comprise eyelets that allow for a suture connection or other form of modular connection with the anchoring frame 96.

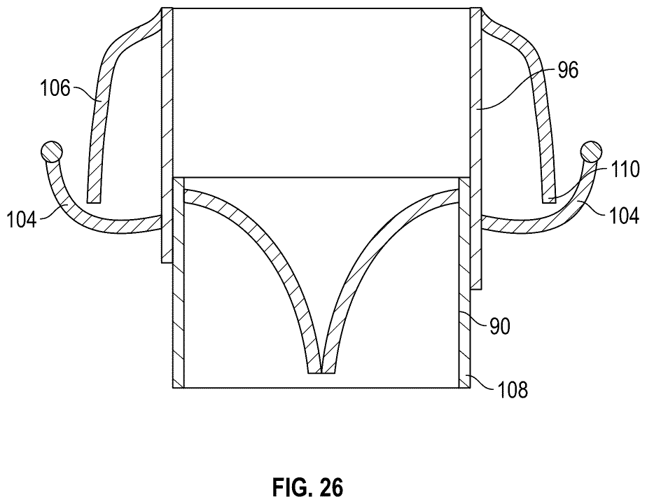

In examples, one or more anchors 104 are coupled to the anchoring frame 96 and protrude radially outward from the anchoring frame 96. The anchors 104 comprise hooks or can have another configuration as disclosed herein. The anchors 104 may be curved elongate anchors and adapted to capture native leaflets between the anchors 104 and the sealing frame 106 (marked in FIG. 26). The anchors 104 are adapted to bend during deployment from an elongated configuration to a hooked, curved, or bent configuration as shown in FIG. 28 for example.

The anchoring frame 96 can be utilized to bear' anchoring load rather than such anchoring load being applied directly to the valve frame 90. The anchors 104 move the native valve leaflets proximal or atrial of the prosthetic valve leaflets to provide an open space for blood flow.

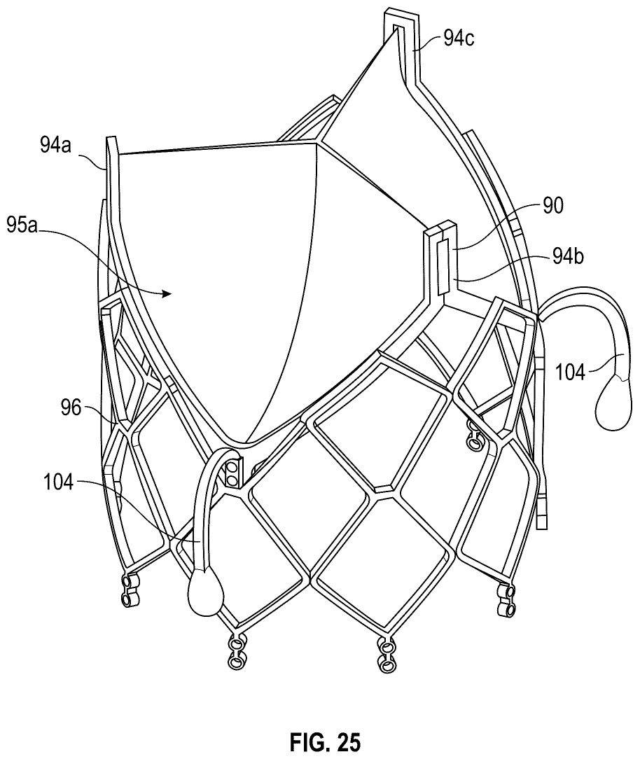

FIG. 22 illustrates a side view of the valve frame 90 coupled to the anchoring frame 96. FIG. 23 illustrates a side view of the valve frame 90. FIG. 24 illustrates a side view of the anchoring frame 96. FIG. 25 illustrates a distal or outflow perspective view of the valve frame 90 coupled to the anchoring frame 96.

The anchoring frame 96 is positioned to protrude in the inflow or proximal direction from the valve frame 90. The anchoring frame 96 uncovers the openings 95a, b, c between the support arms 94a, b, c to allow for blood flow therethrough.

In examples, a sealing frame 106 is coupled to the anchoring frame 96 and surrounds the anchoring frame 96. The sealing frame 106 comprises an outer sealing frame 106 positioned radially outward of the valve frame 90. FIG. 26 illustrates a representative position of the sealing frame 106. The sealing frame 106 can be configured similarly as the sealing frame 22. The sealing frame 106 is configured to engage the native valve. A proximal or inflow portion of the sealing frame 106 couples to a proximal or inflow portion of the anchoring frame 96. The sealing frame 106 is positioned such that the distal end 108 of the valve frame 90 is positioned distal of a distal end 110 of the sealing frame 106. In examples, an outlet end of the valve frame 90 may be positioned further downstream than an outlet end of the sealing frame 106. The openings 95a, b, c of the valve frame 90 accordingly are uncovered by the sealing frame 106, to allow for further blood flow through the openings 95a, b, c. The openings 95a, b, c form blood flow pathways that are uncovered by the sealing frame 106. The anchors 104 of the anchoring frame 96, are positioned proximal or in an inflow direction from the blood flow pathways of the openings 95a, b, c. The prosthetic valve leaflets are positioned in an open space (rather than an enclosed structure) to improve blood flow. In examples, the sealing frame may include a sealing skirt, which may comprise a fabric sealing skirt or other form of skirt. The sealing skirt may be positioned over a substantial portion of the sealing frame 106.

The outlet end of the valve frame 90 is radially spaced apart from the outlet end of the sealing frame 106 for providing the gap therebetween. The outlet end of the valve frame 90 does not contact an outlet end of the sealing frame 106 and a clearance is provided therebetween.

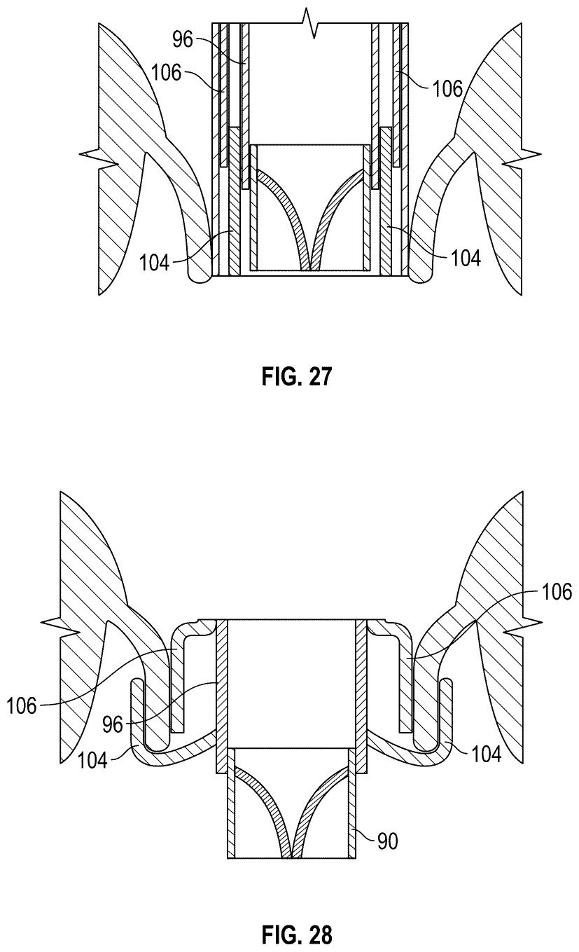

FIGS. 27 and 28 illustrate a representative configuration of the prosthetic valve upon deployment. FIG. 27 illustrates the prosthetic valve in a compressed or undeployed configuration within a capsule of a delivery apparatus. The prosthetic valve can be released from the capsule to position the prosthetic valve in a deployed configuration as shown in FIG. 28. The prosthetic valve can comprise a self-expanding prosthetic valve that expands upon release by the retention body (such as the capsule). The frame of the prosthetic valve (including the valve frame 90, anchoring frame 96, and sealing frame 106) is self-expanding. The frame can be made of a shape memory material that produces the self-expansion. The shape memory material can be Nitinol or another form of shape memory material in examples.

In examples, the prosthetic valve can be configured to be delivered with an inflatable body such as a balloon or other forms of deployment. The inflatable body is positioned within an interior of the prosthetic valve and inflated to expand the prosthetic valve to the expanded configuration.

The valve frame 90 can be positioned sub annular and distal of the position of the anchors 104 and the sealing frame 106. The valve frame 90 is positioned within a native ventricle after deployment. The openings 95a, b, c shown in FIGS. 21 and 23 are uncovered by the sealing frame 106 to allow for blood flow therethrough. A reduced prevalence of blood stagnation results. In examples, the position of the valve frame 90 can be varied to position the valve frame 90 supra annular with respect to the native valve. For example, the valve frame 90 can be positioned at the proximal end portion or inflow end portion of the anchoring frame 96 for supra annular positioning. The openings 95a, b, c comprise open flow channels that are uncovered by the anchoring frame 96 and the sealing frame 106. The prosthetic valve leaflets are positioned in an open space (rather than an enclosed structure) to improve blood flow.

The valve frame 90 emulates a surgical valve structure (e.g., a surgical ring). The sealing frame 106 provides for sealing and the anchoring frame 96 provides for anchoring. This combination of features allows for more physiological flow behavior and thereby reduces the need for sustained anticoagulation therapy. The low thrombogenicity benefit of a bioprosthetic surgical valve is emulated. The prosthetic valve leaflets 92 are suspended in free-space (rather than covered by a cylindrical structure). This type of structure reduces the possibility of thrombus formation due to the open flow channels between the support arms 94a, b, c. The position of the prosthetic valve leaflets 92 in open space reduces the prevalence of turbulence or clotting outside of the prosthetic valve leaflets 92.

The features of FIGS. 21-28 may be utilized solely or in combination with any other example herein.

Various other configurations of prosthetic valves can be utilized in examples.

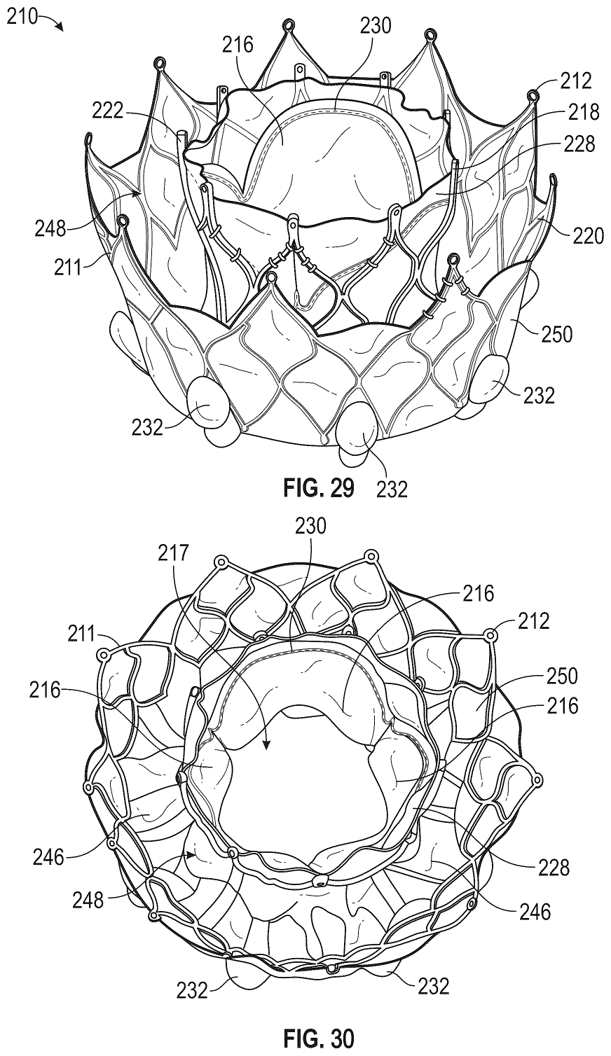

FIG. 29 illustrates a perspective view of a prosthetic valve 210 in the form of a replacement heart valve. The prosthetic valve 210 may be configured to be deployed within a portion of a patient's body. The prosthetic valve 210, for example, may be deployed to an annulus of a native valve, which may comprise a native mitral valve or a native tricuspid valve. In examples other implantation locations may be utilized such as within an aortic or pulmonary valve, or in other valves or locations within a patient's body as desired.

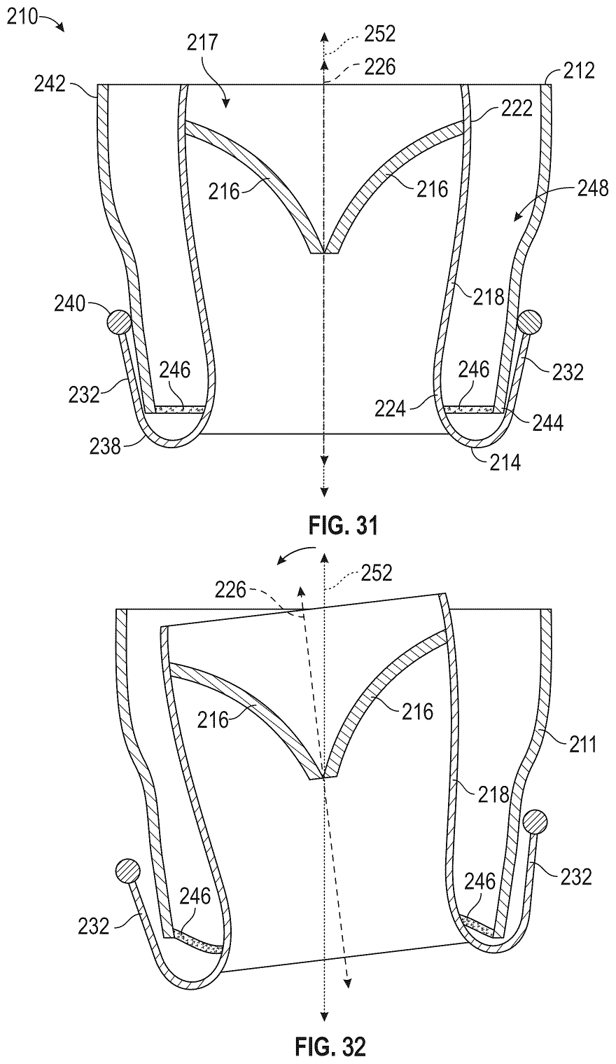

The prosthetic valve 210 may include a proximal end 212 and a distal end 214 (marked in FIG. 31), and a length therebetween. The prosthetic valve 210 may further include a plurality of prosthetic valve leaflets 216 (marked in FIG. 30) configured to surround a flow channel 217 for controlling flow through the valve 210. The prosthetic valve leaflets 216 may be configured to move between opened and closed states to mimic and replace the operation of native valve leaflets.

In examples, the prosthetic valve leaflets 216 may be coupled to a frame. The frame may include an inner frame 218 or valve frame and may include an outer frame 220, which may be part of a sealing body 211.

The inner frame 218 or valve frame may support the plurality of prosthetic valve leaflets 216. The inner frame 218 may have a proximal portion 222 and a distal portion 224 (marked in FIG. 31) and may extend along a first axis 226. The inner frame 218 may include a skirt 228 coupled thereto. The inner frame 218 may surround the flow channel 217.

The inner frame 218 may be contoured in examples such that the proximal portion 222 of the inner frame 218 has a greater diameter than the distal portion 224 of the inner frame 218. The inner frame 218 may contour to curve radially outward in a direction from the distal portion 224 towards the proximal portion 222 of the inner frame 218 (as shown in FIG. 31). The inner frame 218 may be curved or other configurations may be utilized such that the proximal portion 222 has a greater diameter than the distal portion 224. In examples, other configurations of inner frames 218 may be utilized. For example, the inner frame 218 may have a cylindrical shape with a uniform diameter from the proximal portion 222 to the distal portion 224, or may have a curved contour shape with the distal portion 224 having a greater diameter than the proximal portion 222. The proximal portion 222 of the inner frame 218 may flare radially outward in a direction from the distal portion 224 towards the proximal portion 222. Other configurations of inner frames may be utilized in examples.

The prosthetic valve leaflets 216 may couple to the inner frame 218. The prosthetic valve leaflets 216, for example, may couple via a coupling line 230 or suture line that may couple the prosthetic valve leaflets 216 to the skirt 228. The prosthetic valve leaflets 216 may have other forms of coupling in examples.

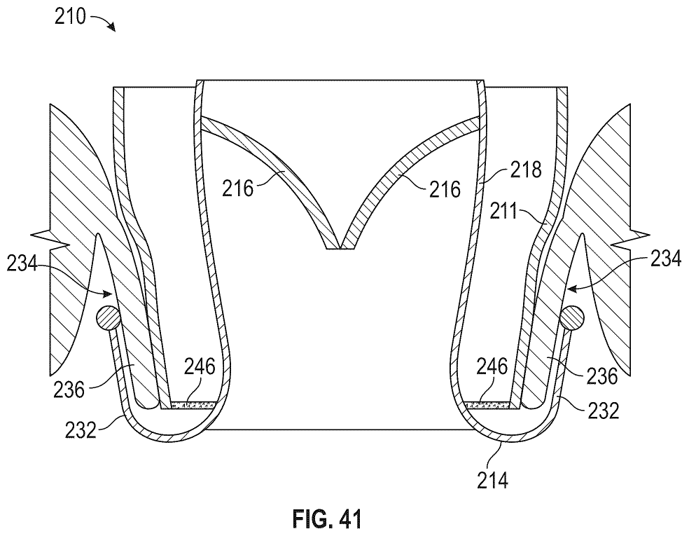

A plurality of anchors 232 may be positioned at the distal portion 224 of the inner frame 218 and may be configured to capture leaflets of a native valve. The anchors 232 may particularly be configured to anchor to the native valve leaflets of the patient's heart. The anchors 232 may extend around the native valve leaflets to anchor to the native valve leaflets. The anchors 232 may be configured to be positioned radially outward of an outward facing surface 234 of a leaflet 236 of the native valve as shown in FIG. 41 for example. The anchors 232 may comprise distal anchors positioned at the distal end 214 of the valve 210, or in examples may be positioned in another position as desired.

The anchors 232 may each extend radially outward from the flow channel 217 and radially outward from the prosthetic valve leaflets 216 of the valve 210. Referring to FIG. 31, the anchors 232 may be coupled to the distal portion 224 of the inner frame 218. The anchors 232 may each include a proximal portion 238 and a distal portion 240, with the proximal portion 238 coupled to the inner frame 218 and the distal portion 240 comprising a tip of the respective anchor 232. The anchors 232 may extend vertically and proximally from the proximal portion 238 to the tip at the distal portion 240 when the valve 210 is deployed.

As shown in FIG. 31, each anchor 232 is configured as a protruding arm configured to extend distally and then curve in a proximal direction to the tip of the respective one of the anchors 232. Such a configuration may allow the anchor 232 to extend around a native leaflet and around the distal tip of the leaflet, to hook around a distal end of the native leaflet and retain the leaflet between the anchor 232 and the sealing body 211. The anchor 232 may be positioned radially outward of an outward facing surface of a leaflet of the native valve. The anchor 232 may thus resist a force applied in the atrial or proximal direction to the valve 210 and may anchor the valve 210 within the native valve annulus. The anchors 232 may be configured to retain the native leaflet between the anchors 232 and the sealing body 211. Other configurations of anchors 232 may be utilized in examples as desired.

The anchors 232 are shown in FIGS. 29-33 in a deployed or expanded configuration, in which the tips of the anchors 232 extend proximally. In examples, the anchors 232 may be configured to be in undeployed, unexpanded, or linearized configuration in which the tips of the anchors 232 extend distally. Upon deployment, the anchors 232 may be configured to move from the undeployed configuration radially outward to the deployed configuration, with the tips flipped towards the proximal direction. Such an operation may allow the anchors 232 to flip over the native valve leaflets to anchor to the native valve leaflets during deployment. Other deployment methods for the anchors 232 may be utilized in examples as desired.

The sealing body 211 may be positioned radially outward from the inner frame 218 and may have a proximal end 242 and a distal end 244 that is coupled to the distal portion 224 of the inner frame 218 such that the sealing body 211 is configured to move with respect to the inner frame 218. In examples, the prosthetic valve 210 may include one or more flexible bodies 246 that may couple the distal end 244 of the sealing body 211 to the distal portion 224 of the inner frame 218 and that allow the sealing body 211 to move with respect to the inner frame 218. The one or more flexible bodies 246 may extend from the inner frame 218 to one or more connection points on the sealing body 211. The one or more flexible bodies 246 may be shaped as arms in examples or may have another shape as desired. The one or more flexible bodies 246 may comprise a fabric or a shape memory material (such as Nitinol) in examples or may have another configuration as desired. The sealing body 211 and the inner frame 218 may lack a direct coupling to each other. The one or more flexible bodies 246 may couple the sealing body 211 to the inner frame 218.

In examples, the one or more flexible bodies 246 may form a covering at the distal end of a gap 248 between the inner frame 218 and the sealing body 211. The one or more flexible bodies 246 may close a distal end portion of the gap 248 positioned between the scaling body 211 and the inner frame 218. The one or more flexible bodies 246, for example, may form a ring positioned at the distal end 244 of the sealing body 211 that connects the distal end 244 of the sealing body 211 to the distal portion 224 of the inner frame 218. The annular ring may form a seal of the distal end 244 of the sealing body 211 such that fluid is impeded from flowing into the gap 248 through the one or more flexible bodies 246.

The sealing body 211 may be configured to seal against a portion of a patient's heart valve. The sealing body 211 may include a frame (which may be an outer frame) 220 and may include a sealing skirt 250, although other configurations may be utilized as desired. The sealing body 211 may be tapered radially inward in a direction from the proximal end 242 towards the distal end 244, although other configurations may be utilized as desired. A proximal end portion of the sealing body 211 may flare radially outward in a direction from the distal end 244 of the sealing body towards the proximal end 242 of the sealing body 211. The sealing body 211 may extend about a second axis 252.

The sealing body 211 may be spaced from the inner frame 218 with the gap 248. The gap 248 may comprise an annular' gap in examples. The proximal end 242 of the sealing body 211 may form an opening for the gap 248. The gap 248 may be closed at the distal end 244 of the sealing body 211. For example, the flexible bodies 246 may comprise a closure of the space between the distal end 244 of the sealing body 211 and the inner frame 218. In an example in which the flexible bodies 246 comprise a cloth material, the cloth material may form a seal of the distal end 244 of the sealing body 211 for example.

The distal end 244 of the sealing body 211 may be coupled to the distal portion 224 of the inner frame 218 such that the sealing body 211 may be configured to move axially with respect to the inner frame 218. For example, the sealing body 211 may move distally along the axis 226 or proximally along the axis 226 relative to the inner frame 218. In examples, the distal end 244 of the sealing body 211 may be coupled to the distal portion 224 of the inner frame 218 such that the sealing body 211 is configured to tilt with respect to the inner frame 218 to offset the first axis 226 from the second axis 252.

FIG. 32, for example, illustrates a tilt of the sealing body 211 with respect to the inner frame 218. In examples, either the sealing body 211 or the inner frame 218 may tilt. The inner frame 218 may tilt in examples, with the scaling body 211 remaining static. Though the inner frame 218 is tilted, the sealing body is not, thus maintaining an optimal seal. The second axis 252 is offset from the first axis 226. The flexible bodies 246 have deflected to allow the sealing body 211 to move with respect to the inner frame 218.

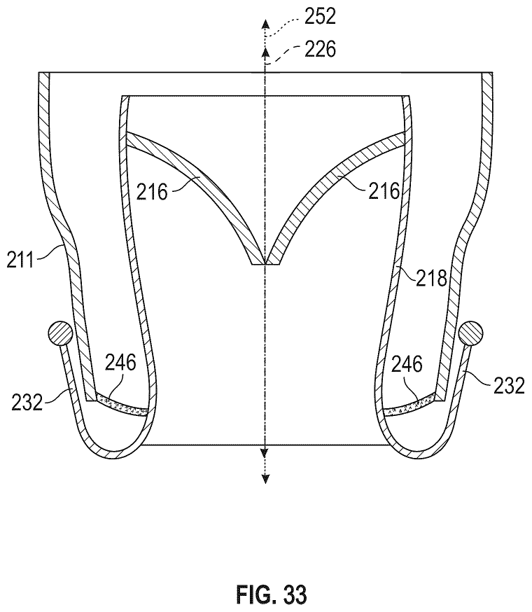

FIG. 33 illustrates an axial movement of the inner frame 218 relative to the sealing body 211. The sealing body 211 is shown to move proximally relative to the inner frame 218, with the one or more flexible bodies 246 deflecting to allow such movement. Combinations of tilting movement and axial movement may be utilized in examples.

The movement may be utilized to accommodate a deployment in which a chord or papillary muscle or the moderator band in a ventricle may be caught by an anchor 232 or another portion of the prosthetic valve. The inner frame 218 as such may tilt or move axially to the position in which it is caught, yet a sealing body 211 may not tilt and may stay with an optimal axis for preventing paravalvular leakage (PVL). Other benefits may be provided by the movement.

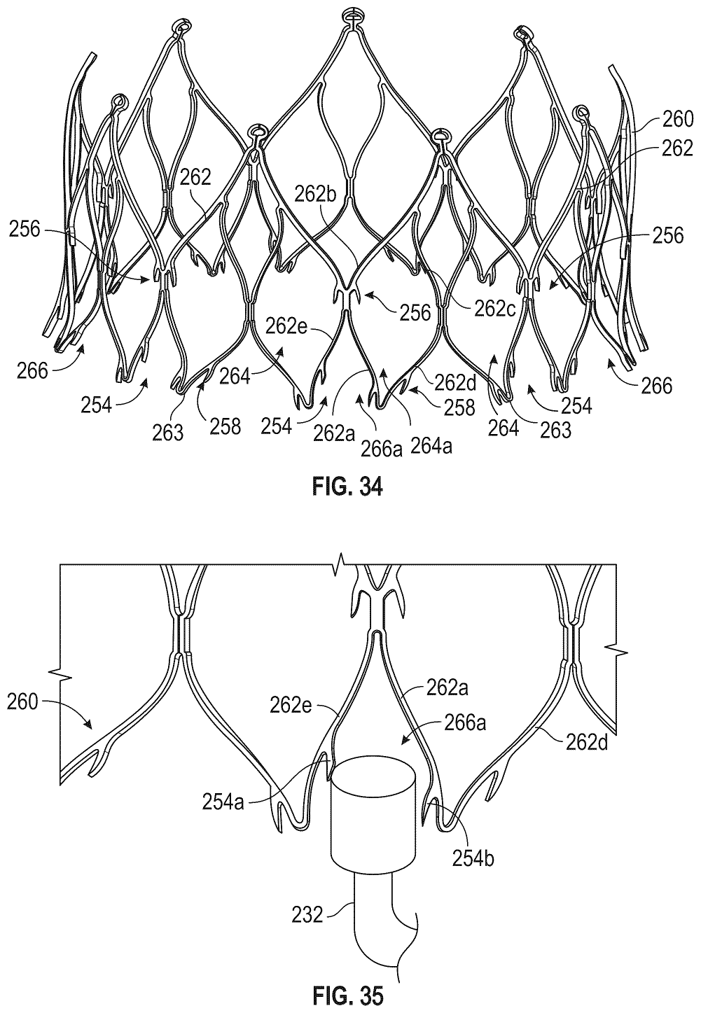

The prosthetic valve 210 may include one or more grip features in examples. The grip features may have a variety of forms. Referring to FIG. 34, for example, the grip features 254, 256, 258 may be positioned on the sealing body and may be positioned on an outer frame 260 of the sealing body. Features of the outer frame 220 may be utilized with the outer frame 260 unless stated otherwise. The grip features 254, 256, 258 may be adapted to engage a portion of the native valve.

The outer frame 260 may include a plurality of struts 262 bounding openings 264, 266 of the outer frame 260. The struts 262 may form a lattice configuration or strut lattice in examples. The struts 262 may form closed cells bounding the openings 264 or may form open cells bounding openings 266. For example, the struts 262 may include the struts 262a-d that may bound the opening 264a. The openings 266 may be unbounded by struts on one portion of the opening 266 (e.g., the openings 266 may be at the distal portion of the outer frame 260 or may form the distalmost openings 266 of the outer frame 260). The grip features 254, 256, 258 may extend towards at least one of the plurality of openings 264, 266 of the outer frame 260 in examples.

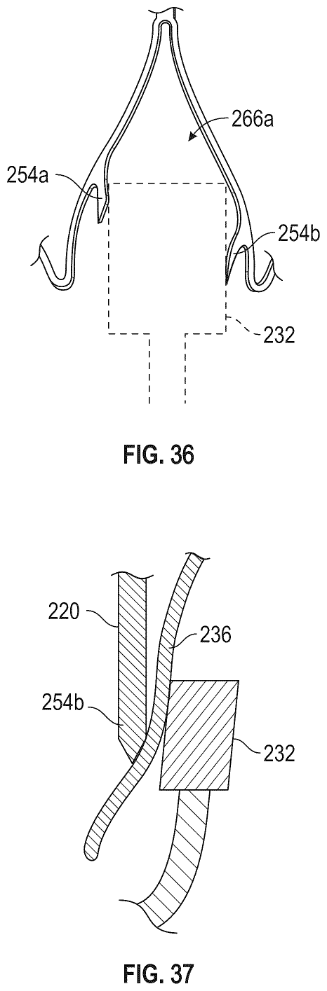

In examples, the openings 266 may align circumferentially with the position of one or more of the anchors 232. Referring to FIG. 35, for example, the openings 266 may include opening 266a, which may be bound by struts 262a, e. The opening 266a may circumferentially align with an anchor 232 such that the anchor 232 may be positioned at the opening 266a. FIG. 36, for example, illustrates the circumferential alignment of the anchor 232 relative to the opening 266a. A tip of the anchor 232 may be positioned at the opening 266a.

The plurality of struts 262 may include distal tips 263 that may flare radially outward in examples.

In examples, the grip features 254 may include grip features 254a, b that may be positioned at the location of the anchor 232. The grip features 254a, b may be configured to engage a surface of the native valve at the position of the anchor 232. The grip features 254a, b, for example, may be positioned such that the anchor 232 may press the native valve against the grip features 254a, b. The anchors 232 may press the native valve against the grip features 254a, b to reduce the possibility of movement of the native valve relative to the outer frame 260. The anchors 232, for example, may press a leaflet against the sealing body and against the grip features 254a, b to grip the leaflet to the grip features 254a, b. In examples, the grip features 254a, b may extend axially or may be flared radially outward. Other configurations may be utilized in examples.

FIG. 37 illustrates an exemplary schematic cross sectional view of a leaflet 236 positioned against the grip feature 254b. The anchor 232 may press the leaflet 236 against the grip feature 254b to cause the grip feature 254b to engage with the leaflet 236. The leaflet 236 may be deflected radially inward or bent at the contact point between the grip feature 254b and the anchor 232. The inward deflection may enhance the engagement between the grip feature 254b and the leaflet 236 and further secure the leaflet 236 to the grip feature 254b.

In examples, one or a plurality of the grip features 254 may be utilized. Referring to FIG. 34, the grip features 254 may be spaced circumferentially to be positioned at locations of anchors 232 in examples. Other positions of grip features 254 may be utilized in examples.

The grip features 254 may reduce movement of the leaflet 236 relative to the sealing body 211. The grip features 254 may enhance securement of the prosthetic valve to an implantation site. The grip features 254, for example, may engage one or more of the leaflets 236 to reduce the possibility of movement of the outer frame 260 proximally or distally relative to the leaflets 236 and secure an orientation of the outer frame 260 to an implantation site. The grip features 254 may reduce a movement of the leaflet 236 relative to the sealing body 211. The grip features 254 may resist a distal movement of the prosthetic heart valve.

The grip features 254 may be engageable due to a deployment procedure of the anchors 232 in examples. For example, the anchors 232 may be extended linearly distal during a deployment procedure and may invert in position to extend proximally in a deployed configuration (as shown in FIGS. 29 and 41 for example). The anchors 232 may invert in position to press against the grip features 254 and secure the leaflet 236 to the grip features 254. A recapture or repositioning procedure may be utilized to extend the anchors 232 linearly and release the leaflet 236 from the grip features 254. The grip features 254 accordingly may be selectively engaged or disengaged by controlling the position of the anchors 232.

Other forms of grip features may be utilized in examples.

The grip features 256, for example, may be configured to engage a portion of the native valve positioned more proximal than the grip features 254. The grip features 256 may be positioned proximal from the grip features 254 on the outer frame 260 in examples. The grip features 256 may be positioned on one or more of the struts 262 and may extend in a direction towards one or more of the openings 264. The grip features 256 may be positioned on strut cells that are closed or may be positioned in other locations as desired.

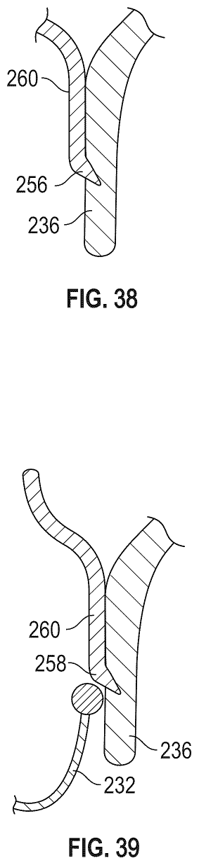

The grip features 256 may be configured to engage a proximal portion of a leaflet 236, or may be configured to engage an annulus of a native valve. FIG. 38, for example, illustrates a grip feature 256 engaging a portion of the leaflet 236. Other positions of engagement may be utilized in examples (e.g., a portion of the annulus of the native valve, or another portion as desired). In examples, the grip features 256 may extend axially or may be flared radially outward.

Other forms of grip features may be utilized in examples.

The grip features 258, for example, may be configured to engage a portion of the native valve in response to one or more of the anchors 232 failing to capture a leaflet 236. The grip features 258, for example, may be configured to flare radially outward such that contact between the grip features 258 with the leaflet 236 may cause the grip features 258 to engage with the leaflet 236. FIG. 39, for example, illustrates such a configuration. The anchor 232 has failed to capture the leaflet 236. The anchor 232 accordingly is positioned radially inward of the mis-captured leaflet 236. The grip features 258 may engage a surface of the native valve leaflet upon the anchor 232 failing to capture the leaflet 236. The grip features 258 may protrude radially outward from the anchor 232 and may engage with an interior surface of the leaflet 236. The grip features 258 may accordingly secure the outer frame 260 in position relative to the leaflet 236 and impede proximal or distal movement of the outer frame 260 relative to the leaflet 236. The outer frame 260 may form a seal with the leaflet 236 to reduce the possibility of fluid flow radially outward of the outer frame 260 and radially outward of the prosthetic valve (e.g., paravalvular leakage).

The grip features 254, 256, 258 may have a variety of configurations. The grip features 254, 256, 258 may comprise barbs in examples, which may be configured to penetrate through tissue of the native heart valve (e.g., the native heart valve leaflets) to engage with the tissue. The barbs may extend distally or may have other orientations in examples. Other configurations of grip features 254, 256, 258 may be utilized in examples.

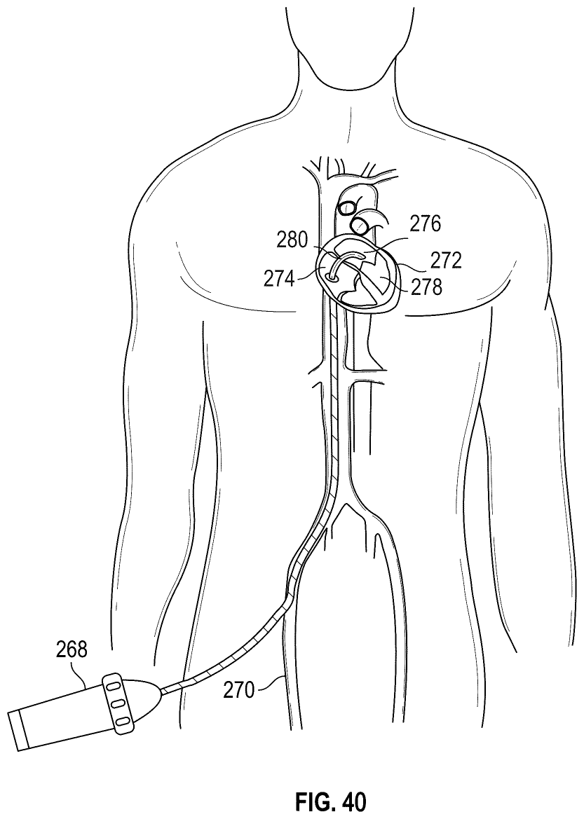

FIGS. 40 and 41 illustrate an exemplary deployment sequence of the prosthetic valve 210. The prosthetic valve 210 may include any of the features disclosed in regard to FIGS. 29-39, including use of one or more grip features. A delivery apparatus 268 may be utilized to approach an implantation site. The delivery apparatus 268 may be passed percutaneously into a patient's body in a minimally invasive manner. In other examples, more invasive means may be utilized as desired.

The delivery apparatus 268 may be utilized for transcatheter delivery of the valve. The delivery apparatus 268 may pass transvenous through the femoral vein 270 or another portion of the patient's vasculature. For example, transjugular entry or other methods of entry may be utilized as desired. The delivery apparatus 268 may pass to the patient's heart 272.

The delivery apparatus 268 may be used to deliver the valve to the tricuspid valve, and as such, may be positioned within the right atrium 274 of the patient's heart for delivery to the tricuspid valve. In an example in which delivery is to the mitral valve, the delivery apparatus 268 may pass transseptal to the left atrium 276 for delivery to the mitral valve. The delivery apparatus 268 may advance towards the left ventricle 278 of the patient's heart for mitral delivery.

The prosthetic valve 210 may be retained in a compressed or undeployed configuration within a capsule 280 of the delivery apparatus 268. The prosthetic valve 210 may be released from the capsule 280 at the implantation site. FIG. 41 illustrates a deployed configuration of the prosthetic valve. The anchors 232 may be released from the capsule 280 to be positioned radially outward of the native valve leaflet 236. The sealing body 211 may be released from the capsule 280 to expand radially outward and contact the native valve (e.g., the inward facing surfaces of the native valve leaflets 236). In examples, one or more of the grip features 254, 256, 258 may be utilized for securement of the sealing body 211 in a desired position relative to the native valve (e.g., relative to the native valve leaflets 236).

The inner frame 218 may be connected to the sealing body 211 with the one or more flexible bodies 246. The inner frame 218 accordingly may be able to tilt or move axially with respect to the sealing body 211 to conform to a desired shape of the implantation site. Improved sealing and/or remodeling of the native valve may result.

In examples, the outer frame 260 of the sealing body 211 may be conformable to allow the outer frame 260 to conform to a shape of the native valve. For example, a native valve having an irregular shape or “D” shape may be conformed to with the outer frame 260. The inner frame 218 may maintain a symmetrical or uniform shape with the outer frame 260 having an irregular shape in examples. The outer frame 260 may seal with the native valve.

The features of FIGS. 29-41 may be utilized solely or in combination with any other example herein.

Other configurations of the prosthetic valve 210 may be utilized in examples.

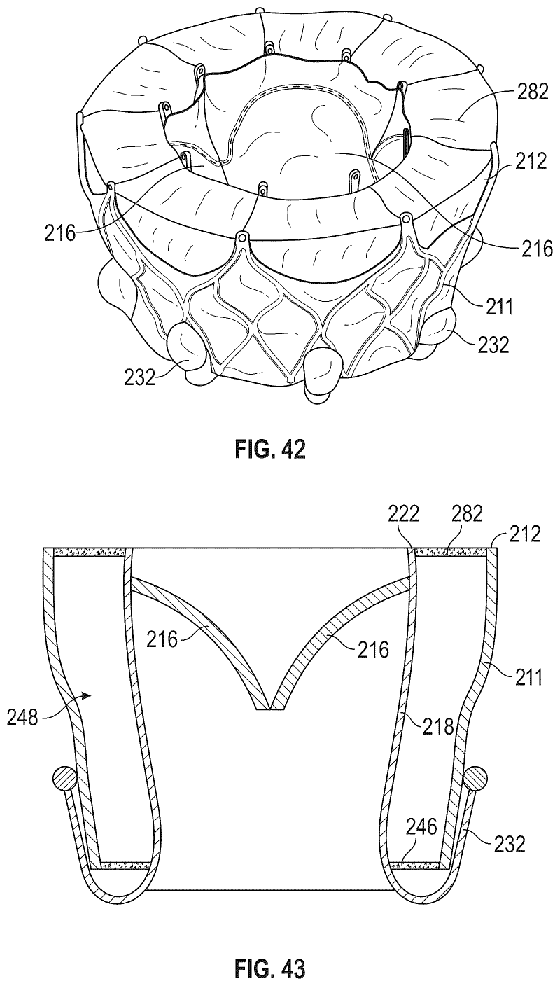

FIGS. 42 and 43, for example, illustrates an example in which one or more flexible bodies 282 may extend from the proximal end 212 of the sealing body 211 to the proximal portion 222 of the inner frame 218. The flexible bodies 282 may have a similar' construction as the one or more flexible bodies 246. For example, the flexible bodies 282 may be configured to seal fluid flow therethrough to accordingly seal a proximal end of the gap 248. The flexible bodies 282 may be flexible to allow for movement of the sealing body 211 relative to the inner frame 218 (e.g., a tilt or axial movement of the sealing body 211 relative to the inner frame 218). FIG. 42 illustrates a perspective view of a prosthetic valve having the proximal end of the gap 248 closed, and FIG. 43 illustrates a cross sectional schematic view of the prosthetic valve. The one or more flexible bodies 282 may comprise a fabric bridge that extends to close the proximal end portion of the gap 248.

The features of FIGS. 42 and 43 may be utilized solely or in combination with any other example herein.

Other configurations of prosthetic valves may be utilized in examples.

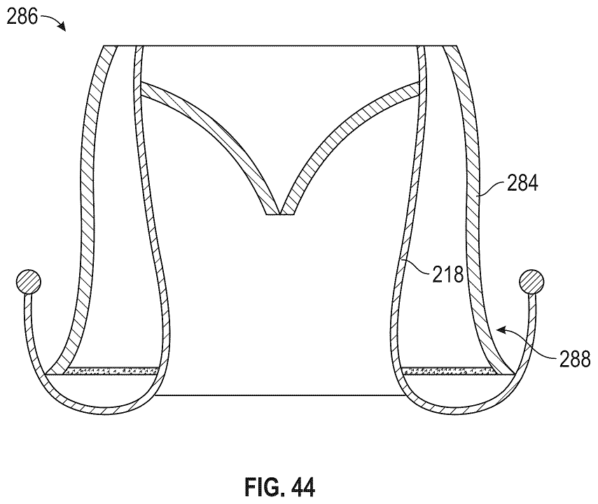

FIG. 44, for example, illustrates an example in which an outer frame 284 of a sealing body curves or tapers radially inward at a proximal portion 286 of the outer frame 284 in a direction from the distal portion of the outer frame 284 towards the proximal portion of the outer frame 284. The distal portion 288 of the outer frame 284 may curve or taper radially outward at a distal portion 288 of the outer frame 284 in a direction from the proximal portion 286 of the outer frame 284 in a direction towards the distal portion 288 of the outer frame 284. The inner frame 218 may have a similar configuration as discussed in regard to FIG. 29-31 or may have another configuration in examples as desired.

The features of FIG. 44 may be utilized solely or in combination with any other example herein.

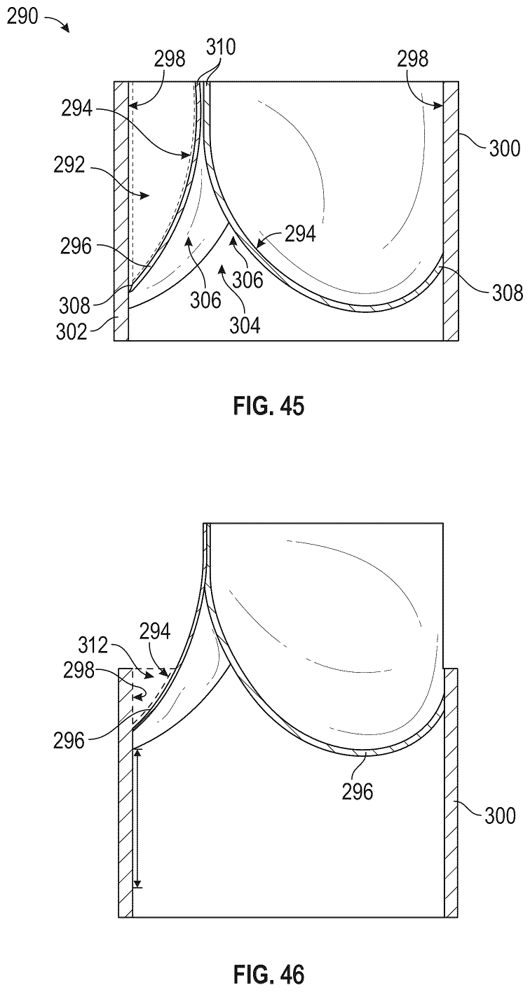

In examples, configurations of prosthetic valve leaflets may be utilized. Such configurations of prosthetic valve leaflets may be utilized to reduce stagnation of fluid (e.g., blood) flow at a neo-sinus space of a prosthetic valve. FIG. 45, for example, comprises an exemplary diagram of a prosthetic valve 290 illustrating a position of a neo-sinus space 292 (marked by dashed lines in FIG. 45). The neo-sinus space 292 may be positioned between the outward facing surface 294 of one of the prosthetic valve leaflets 296 and an inward facing surface 298 of a valve body 300 supporting the prosthetic valve leaflets 296. The valve body 300, for example, may be configured to impede fluid flow through a wall 302 of the valve body 300, through use of a sealing skirt or through another configuration of the wall 302 that impedes fluid flow therethrough. The neo-sinus space 292 accordingly may comprise a space or pocket in which fluid may be retained.

The neo-sinus space 292 may comprise a space or pocket in which fluid may be stagnant upon the opening or closing of the prosthetic valve leaflets 296. The prosthetic valve leaflets 296 may be positioned about a flow channel 304 for fluid flow therethrough. The inward facing surfaces 306 of the prosthetic valve leaflets 296 may be configured to face towards the flow channel 304, with the outward facing surfaces 294 facing opposite the inward facing surfaces 306 and accordingly opposite the flow channel 304. The neo-sinus space 292 may have a reduced fluid flow therethrough because the fluid flowing through the flow channel 304 in a direction from an inflow edge 308 of the prosthetic valve leaflets 296 towards an outflow edge 310 of the prosthetic valve leaflets 296 may be impeded from passing to the neo-sinus space 292 by the prosthetic valve leaflets 296. As such, lesser flow to the neo-sinus space 292 may result in stagnation of fluid (e.g., blood), which may produce adverse results (e.g., thrombosis formation, among other adverse results).

Repositioning the prosthetic valve leaflets 296 may yet produce a neo-sinus space 312. FIG. 46, for example, illustrates a repositioning of the leaflets 296 in an outflow direction relative to the position shown in FIG. 45. The neo-sinus space 312 may exist between the outward facing surface 294 of one of the prosthetic valve leaflets 296 and the inward facing surface 298 of the valve body 300 supporting the prosthetic valve leaflets 296. The neo-sinus space 312 may have a smaller size than the space 292 shown in FIG. 45, yet may produce stagnation of fluid (e.g., blood) within the space 312.

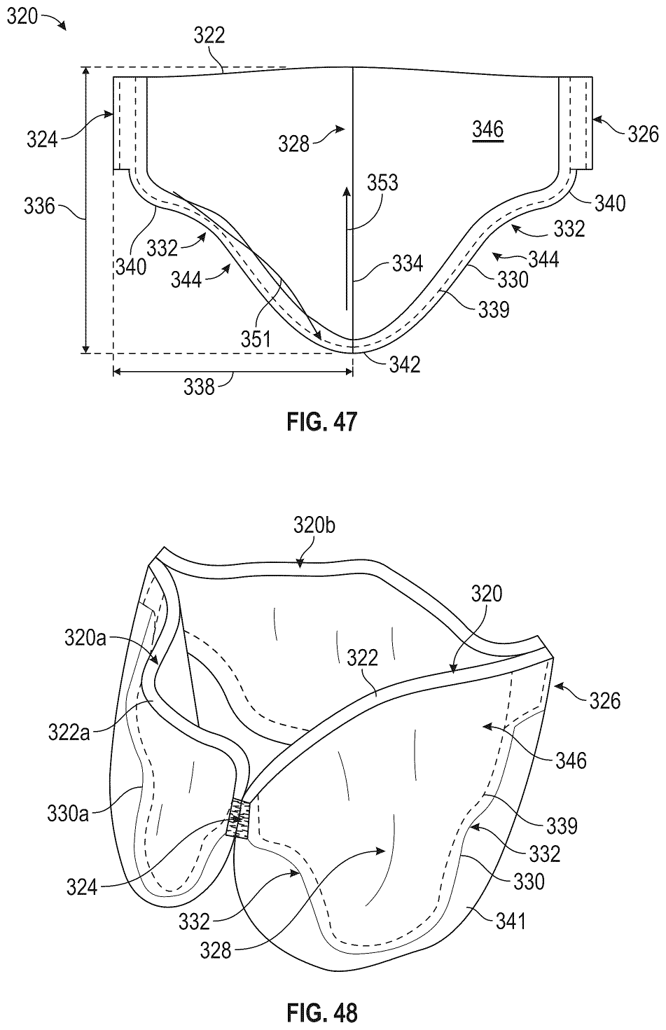

FIGS. 47 and 48 illustrate an example of a prosthetic valve leaflet 320 that may be utilized in examples herein. A flat pattern of the prosthetic valve leaflet 320 is shown in FIG. 47. Referring to FIG. 47, the prosthetic valve leaflet 320 may include an outflow edge 322, a first side commissure end 324, and a second side commissure end 326 positioned opposite the first side commissure end 324. The first side commissure end 324 and the second side commissure end 326 may both extend axially in examples. The prosthetic valve leaflet 320 may include a central portion 328 positioned between the first side commissure end 324 and the second side commissure end 326.

The prosthetic valve leaflet 320 may include an inflow edge 330 positioned opposite the outflow edge 322. In examples, the inflow edge 330 may include at least one indentation 332 towards the central portion 328. The indentation 332 may comprise a concave curvature in the inflow edge 330 or may have other configurations as desired. As shown in FIG. 47, an indentation 332 may be positioned on the inflow edge 330 between a mid-line 334 of the leaflet 320 and the first side commissure end 324 of the leaflet 320, and an indentation 332 may be positioned on the inflow edge 330 between the mid-line 334 of the leaflet 320 and the second side commissure end 326 of the leaflet 320. The indentations 332 may be positioned symmetrical about the mid-line 334 or may have other positions as desired. In examples, a single indentation, or a number of indentations greater than two may be utilized in examples.

Referring to FIG. 47, one or more of the indentations 332 may be positioned at least 40% of the distance 336 along the prosthetic valve leaflet 320 from the outflow edge 322 towards the inflow edge 330. In examples, one or more of the indentations 332 may have other positions (e.g., at least 30% of the distance 336 along the prosthetic valve leaflet 320 from the edge 322 towards the edge 330, at least 50%, at least 60%, at least 70%, etc.). At least two indentations may be positioned symmetrical about the axially extending mid-line 334 or may have asymmetrical positioning in examples.

One or more of the indentations 332 may be positioned at least 60% of the distance 338 along the prosthetic valve leaflet 320 from the mid-line 334 towards the first side commissure end 324. Another of the indentations 332 may similarly be positioned at least 60% of the distance along the prosthetic valve leaflet 320 from the mid-line 334 towards the second side commissure end 326. Other percentages from the mid-line 334 towards a respective one of the first side commissure end 324 or the second side commissure end 326 may be utilized (e.g., at least 40%, at least 50%, at least 70%, at least 80%, etc.).

The inflow edge 330 may include an outer portion 340, an inner portion 342, and a mid portion 344 between the outer portion 340 and the inner portion 342. The outer portion 340, in examples, may be convex with respect to the central portion 328. The inner portion 342, in examples, may be convex with respect to the central portion 328. The mid portion 344 may comprise the portion of the inflow edge 330 including the one or more indentations 332 in examples. The inner portion 342 and outer portion 340 may have parabolic shapes or other curvatures bowing in an inflow direction, and the one or more indentations may have parabolic shapes or other curvatures bowing towards the central portion 328 in examples.

In examples, the inflow edge 330 may comprise a coupling line 339 that may be utilized to couple the leaflet 320 to other portions of a leaflet support structure such as a skirt 341 (marked in FIG. 48) or other support material. The coupling line 339 may comprise a suture line in examples that may be utilized to suture the leaflets 320 to the skirt 341 or other support material. The suture line may follow the contour of the inflow edge 330 and may have the shape of the inflow edge 330 in examples.

In examples, the respective side commissure ends 324, 326 may include coupling lines for coupling the leaflets 320 to a support material (such as a skirt) or directly to an adjacent leaflet 320a, b (as marked in FIG. 48). The respective side commissure ends 324, 326 may comprise the commissures of the adjacent leaflets 320, 320a, 320b (as shown in FIG. 48).

The respective side commissure ends 324, 326 may comprise tabs in examples that may be utilized for coupling of adjacent leaflets 320, 320a, 320b to each other. The tabs may further be configured for coupling to a frame or insertion into apertures of a frame in examples.

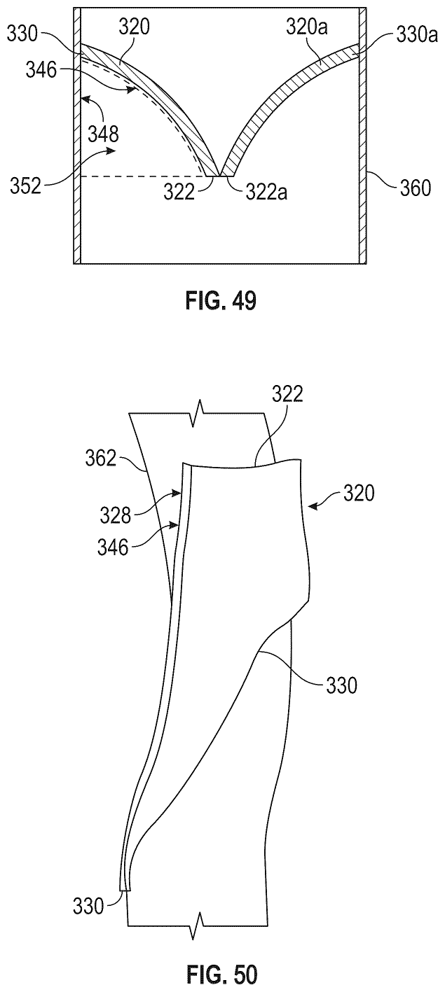

The one or more indentations 332 may reduce the size of a neo-sinus space in examples. Referring to FIG. 47, the one or more indentations 332 may reduce a size of an area positioned between the outward facing surface 346 of the leaflet 320 and an inward facing surface 348 of the valve body 350 (marked in FIG. 49). A reduced size of the neo-sinus space 352 (marked in FIG. 49) may reduce the possibility of stagnation of fluid in the closed area comprising the neo-sinus space 352 and reduce the possibility of adverse results from stagnation in the neo-sinus space 352 (e.g., formation of thrombosis or other adverse results). Further, the one or more indentations 332 may direct fluid flow towards the inner portion 342 of the leaflet 320 as indicated by the flow arrow 351 in FIG. 47. The fluid flow toward the inner portion 342 may clear out fluid at the inner portion 342 during the systolic and diastolic movement of the leaflet 320. The fluid may be directed towards the outflow edge 322 as represented by the flow arrow 353 in FIG. 47. As such, stagnation of fluid at the inner portion 342 and otherwise in the neo-sinus space 352 may be reduced.

FIG. 48 illustrates an arrangement of three prosthetic valve leaflets 320, 320a, 320b coupled together. The leaflets 320a, b may each be configured similarly as the leaflet 320. The respective side commissure ends of the prosthetic valve leaflets 320, 320a, 320b may be joined together. Each of the prosthetic valve leaflets 320, 320a, 320b may be coupled to a respective skirt 341 that may be utilized to seal flow or couple the leaflets 320, 320a, 320b to a frame or other portion of a valve body. In examples, three prosthetic valve leaflets 320, 320a, 320b or a lesser number (e.g., two) or a greater number of prosthetic valve leaflets may be utilized as desired. The outflow edges 322 of the leaflets 320, 320a, 320b may comprise coaptation surfaces in examples.

FIG. 49 illustrates a schematic view of the leaflets 320, 320a in position within a valve body 360. The respective inflow edges 330a may couple to the valve body 360 with the respective outflow edges 322, 322a extending in an outflow direction. The valve body 360 may have a cylindrical shape in examples or may have another shape as desired.

In examples, the valve body 360 may curve radially inward. FIG. 50, for example, illustrates a cross sectional view of a portion of a valve body 362 having a radially inward curvature. The leaflet 320 is shown in cross sectional view in an opened configuration (i.e., valve leaflets opened to allow fluid flow through the flow channel). The outward facing surface 346 of the leaflet 320 may contour to the shape of the valve body 360 in the opened configuration to close a portion of the neo-sinus space at the inner portion 342 of the prosthetic leaflet 320 and thus eject fluid from this portion of the neo-sinus space during opening of the leaflets. As such, reduced possibility of stagnation due to the neo-sinus space may result.

In examples, a varied stitch or suture pattern may be utilized to produce a reduced size of the neo-sinus space in examples.

The features of FIGS. 47-50 may be utilized solely or in combination with any other example herein.

Various modifications of the examples disclosed herein can be provided. Features of examples can be modified, substituted, excluded, or combined across examples as desired. Combinations of features across examples can be provided as desired. Combinations of features can be provided across examples with other features of such examples being excluded if desired.

The various examples of sealing skirts disclosed herein can have a variety of forms, including cloth skirts, foam skirts, or braided skirts as desired. Various materials can be utilized as desired.

The various examples of prosthetic valve leaflets can be made from a variety of materials as desired. The prosthetic valve leaflets can be made from pericardium, preferably bovine or equine. Other materials can be utilized, e.g., polymers, among others.

The implants disclosed herein can include prosthetic heart valves or other forms of implants, such as stents or filters, or diagnostic devices, among others. The implants can be expandable implants configured to move from a compressed or undeployed state to an expanded or deployed state. The implants can be compressible implants configured to be compressed inward to have a reduced outer profile and to move the implant to the compressed or undeployed state.

Various forms of delivery apparatuses can be utilized with the examples disclosed herein. The delivery apparatuses as disclosed herein can be utilized for aortic, mitral, tricuspid, and pulmonary replacement and repair as well. The delivery apparatuses can comprise delivery apparatuses for delivery of other forms of implants, such as stents or filters, or diagnostic devices, among others.

The implants and the systems disclosed herein can be used in transcatheter mitral or tricuspid implantation, as well as aortic valve implantation (TAVI) or replacement of other native heart valves (e.g., pulmonary valves). The delivery apparatuses and the systems disclosed herein can be utilized for transarterial access, including transfemoral access, to a patient's heart. The delivery apparatuses and systems can be utilized in transcatheter percutaneous procedures, including transarterial procedures. Transfemoral or transjugular access can be utilized. Transapical procedures, among others, can also be utilized. Other procedures can be utilized as desired.

In addition, the methods herein are not limited to the methods specifically described, and can include methods of utilizing the systems and apparatuses disclosed herein. The steps of the methods can be modified, excluded, or added to, with systems, apparatuses, and methods disclosed herein. The examples disclosed herein can comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present, or problems be solved. Features, elements, or components of one example can be combined into other examples herein.

Example 1: A prosthetic valve for deployment to a native valve, the prosthetic valve comprising: a plurality of prosthetic valve leaflets positioned about a flow channel of the prosthetic valve and having a plurality of commissures; a valve frame for supporting the plurality of prosthetic valve leaflets and being expandable from a compressed configuration radially outward to an expanded configuration, the valve frame including: a first support arm extending axially and supporting at least two of the plurality of prosthetic valve leaflets at a first commissure of the plurality of commissures, a second support arm extending axially and supporting at least two of the plurality of prosthetic valve leaflets at a second commissure of the plurality of commissures, and an opening positioned circumferentially between the first support arm and the second support arm and for allowing for blood flow therethrough; and a sealing frame for sealing with the native valve.

Example 2: The prosthetic valve of any example herein, in particular Example 1, wherein a first prosthetic valve leaflet of the plurality of prosthetic valve leaflets includes a first commissure end portion positioned at the first commissure and a second commissure end portion positioned at the second commissure and a central portion positioned between the first commissure end portion and the second commissure end portion, and the opening is circumferentially aligned with the central portion of the first prosthetic valve leaflet.

Example 3: The prosthetic valve of any example herein, in particular Example 1 or Example 2, wherein the opening spans from the first support arm to the second support arm.

Example 4: The prosthetic valve of any example herein, in particular Examples 1-3, wherein the opening has a “U” shape.

Example 5: The prosthetic valve of any example herein, in particular Examples 1-4, wherein the first support arm extends axially in an outflow direction to a free end of the first support arm.

Example 6: The prosthetic valve of any example herein, in particular Examples 1-5, wherein the valve frame includes a third support arm extending axially and supporting at least two of the plurality of prosthetic valve leaflets at a third commissure of the plurality of commissures.

Example 7: The prosthetic valve of any example herein, in particular Example 6, wherein the first support arm, the second support arm, and the third support arm are spaced circumferentially equidistant from each other.

Example 8: The prosthetic valve of any example herein, in particular Examples 1-7, further comprising a sealing skirt positioned on the sealing frame.

Example 9: The prosthetic valve of any example herein, in particular Example 8, wherein the sealing frame protrudes outward radially from the valve frame.

Example 10: The prosthetic valve of any example herein, in particular Examples 1-9, wherein a proximal portion of the prosthetic valve comprises an inflow portion and a distal portion of the prosthetic valve comprises an outflow portion, and the scaling frame includes a proximal portion that protrudes radially outward from a proximal end of the sealing frame.

Example 11: The prosthetic valve of any example herein, in particular Example 10, wherein the sealing frame includes a distal portion that extends in a distal direction.

Example 12: The prosthetic valve of any example herein, in particular Example 11, wherein the sealing frame includes a curved portion positioned between the proximal portion and the distal portion.

Example 13: The prosthetic valve of any example herein, in particular Examples 1-12, wherein the scaling frame surrounds the first support arm and the second support arm.

Example 14: The prosthetic valve of any example herein, in particular Examples 1-13, wherein the sealing frame is spaced from the valve frame with a gap.

Example 15: The prosthetic valve of any example herein, in particular Example 14, wherein the gap forms at least a portion of a ring extending about the valve frame.

Example 16: The prosthetic valve of any example herein, in particular Example 14 or Example 15, further comprising one or more flow barriers positioned within the gap.

Example 17: The prosthetic valve of any example herein, in particular Examples 1-16, wherein a proximal portion of the prosthetic valve comprises an inflow portion and a distal portion of the prosthetic valve comprises an outflow portion, and a proximal portion of the valve frame is coupled to a proximal portion of the sealing frame.

Example 18: The prosthetic valve of any example herein, in particular Example 17, wherein a distal portion of the sealing frame is spaced from the valve frame to form an opening for an interior of the sealing frame.

Example 19: The prosthetic valve of any example herein, in particular Examples 1-18, wherein a proximal portion of the prosthetic valve comprises an inflow portion and a distal portion of the prosthetic valve comprises an outflow portion, and a distal end of the valve frame is positioned distal of a distal end of the sealing frame.

Example 20: The prosthetic valve of any example herein, in particular Examples 1-19, wherein the opening forms a blood flow pathway that is uncovered by the sealing frame.

Example 21: The prosthetic valve of any example herein, in particular Examples 1-20, wherein the plurality of prosthetic valve leaflets are positioned to be retained sub annular or supra annular with respect to the native valve.

Example 22: The prosthetic valve of any example herein, in particular Examples 1-21, further comprising an anchoring frame modularly coupled to the valve frame.

Example 23: The prosthetic valve of any example herein, in particular Example 22, wherein the anchoring frame comprises a lattice structure of struts and openings.

Example 24: The prosthetic valve of any example herein, in particular Example 22 or Example 23, wherein the anchoring frame has a cylindrical shape.

Example 25: The prosthetic valve of any example herein, in particular Examples 1-24, further comprising one or more anchors for anchoring the prosthetic valve to the native valve.

Example 26: The prosthetic valve of any example herein, in particular Example 25, wherein the one or more anchors comprise hooks.

Example 27: The prosthetic valve of any example herein, in particular Example 25 or Example 26, wherein the one or more anchors are shaped to extend over a distal tip of a native valve leaflet of the native valve.

Example 28: The prosthetic valve of any example herein, in particular Examples 25-27, wherein the one or more anchors extend from one or more of the first support arm or the second support arm.

Example 29: The prosthetic valve of any example herein, in particular Examples 25-28, wherein the one or more anchors extend from the sealing frame.

Example 30: The prosthetic valve of any example herein, in particular Examples 25-29, further comprising an anchoring frame modularly coupled to the valve frame, and wherein the one or more anchors extend from the anchoring frame.

Example 31: The prosthetic valve of any example herein, in particular Example 30, wherein a proximal portion of the prosthetic valve comprises an inflow portion and a distal portion of the prosthetic valve comprises an outflow portion, and wherein the one or more anchors are positioned proximal of a blood flow pathway formed by the opening.

Example 32: The prosthetic valve of any example herein, in particular Examples 1-31, wherein the sealing frame comprises a lattice structure of struts and openings.

Example 33: The prosthetic valve of any example herein, in particular Examples 1-32, wherein the sealing frame is expandable from a compressed configuration radially outward to an expanded configuration.

Example 34: The prosthetic valve of any example herein, in particular Examples 1-33, wherein the prosthetic valve is adapted for transcatheter delivery to the native valve.

Example 35: The prosthetic valve of any example herein, in particular Examples 1-34, wherein the prosthetic valve comprises a prosthetic mitral heart valve or a prosthetic tricuspid heart valve.

Example 36: A method comprising: deploying a prosthetic valve to a native valve, the prosthetic valve including: a plurality of prosthetic valve leaflets positioned about a flow channel of the prosthetic valve and having a plurality of commissures, a valve frame for supporting the plurality of prosthetic valve leaflets and being expandable from a compressed configuration radially outward to an expanded configuration, the valve frame including: a first support arm extending axially and supporting at least two of the plurality of prosthetic valve leaflets at a first commissure of the plurality of commissures, a second support arm extending axially and supporting at least two of the plurality of prosthetic valve leaflets at a second commissure of the plurality of commissures, and an opening positioned circumferentially between the first support arm and the second support arm and for allowing for blood flow therethrough, and a sealing frame for sealing with the native valve.

Example 37: The method of any example herein, in particular' Example 36, wherein a first prosthetic valve leaflet of the plurality of prosthetic valve leaflets includes a first commissure end portion positioned at the first commissure and a second commissure end portion positioned at the second commissure and a central portion positioned between the first commissure end portion and the second commissure end portion, and the opening is circumferentially aligned with the central portion of the first prosthetic valve leaflet.

Example 38: The method of any example herein, in particular Example 36 or Example 37, wherein the opening has a “U” shape.

Example 39: The method of any example herein, in particular Examples 36-38, wherein the valve frame includes a third support arm extending axially and supporting at least two of the plurality of prosthetic valve leaflets at a third commissure of the plurality of commissures.

Example 40: The method of any example herein, in particular Examples 36-39, wherein a proximal portion of the prosthetic valve comprises an inflow portion and a distal portion of the prosthetic valve comprises an outflow portion, and the sealing frame includes a proximal portion that protrudes radially outward from a proximal end of the sealing frame.

Example 41: The method of any example herein, in particular Examples 36-40, wherein the scaling frame surrounds the first support aim and the second support arm.

Example 42: The method of any example herein, in particular Examples 36-41, wherein a proximal portion of the prosthetic valve comprises an inflow portion and a distal portion of the prosthetic valve comprises an outflow portion, and a proximal portion of the valve frame is coupled to a proximal portion of the sealing frame.

Example 43: The method of any example herein, in particular Examples 36-42, wherein a proximal portion of the prosthetic valve comprises an inflow portion and a distal portion of the prosthetic valve comprises an outflow portion, and a distal end of the valve frame is positioned distal of a distal end of the sealing frame.

Example 44: The method of any example herein, in particular Examples 36-43, wherein an anchoring frame is modularly coupled to the valve frame.

Example 45: The method of any example herein, in particular Examples 36-44, wherein the native valve is a mitral heart valve or a tricuspid heart valve.

Example 46: A prosthetic valve for deployment to a native valve, the prosthetic valve comprising: a plurality of prosthetic valve leaflets; an inner frame supporting the plurality of prosthetic valve leaflets and having a proximal portion and a distal portion; a plurality of anchors positioned at the distal portion of the inner frame and adapted to couple to the native valve; a sealing body positioned radially outward from the inner frame and having a proximal end and a distal end that is coupled to the distal portion of the inner frame such that the sealing body is adapted to move with respect to the inner frame; and one or more grip features positioned on the sealing body and adapted to engage a portion of the native valve.

Example 47: The prosthetic valve of any example herein, in particular Example 46, wherein one or more flexible bodies couple the distal end of the sealing body to the distal portion of the inner frame and allow the sealing body to move with respect to the inner frame.

Example 48: The prosthetic valve of any example herein, in particular Example 47, wherein the one or more flexible bodies comprise a fabric or a shape memory material.

Example 49: The prosthetic valve of any example herein, in particular Example 47 or Example 48, wherein the one or more flexible bodies form a ring positioned at the distal end of the sealing body.

Example 50: The prosthetic valve of any example herein, in particular Examples 47-49, wherein the one or more flexible bodies close a distal end portion of an annular gap positioned between the sealing body and the inner frame.

Example 51: The prosthetic valve of any example herein, in particular Examples 46-50, wherein the inner frame extends about a first axis and the sealing body extends about a second axis, and the distal end is coupled to the distal portion of the inner frame such that the sealing body is adapted to tilt with respect to the inner frame to offset the first axis from the second axis.

Example 52: The prosthetic valve of any example herein, in particular Examples 46-51, wherein the distal end is coupled to the distal portion of the inner frame such that the scaling body is adapted to move axially with respect to the inner frame.

Example 53: The prosthetic valve of any example herein, in particular Examples 46-52, wherein the sealing body is spaced from the inner frame with an annular gap and the proximal end of the sealing body forms an opening for the annular gap.

Example 54: The prosthetic valve of any example herein, in particular Examples 46-53, wherein the sealing body is spaced from the inner frame with an annular gap and a fabric bridge extends from the proximal end of the sealing body to the proximal portion of the inner frame to close a proximal end portion of the annular gap.

Example 55: The prosthetic valve of any example herein, in particular Examples 46-54, wherein the inner frame comprises a first frame and the sealing body includes a second frame and a sealing skirt.

Example 56: The prosthetic valve of any example herein, in particular Examples 46-55, wherein the scaling body is tapered radially inward in a direction from the proximal end towards the distal end.

Example 57: The prosthetic valve of any example herein, in particular Examples 46-56, wherein a proximal end portion of the sealing body flares radially outward in a direction from the distal end of the sealing body towards the proximal end of the sealing body.

Example 58: The prosthetic valve of any example herein, in particular Examples 46-57, wherein the proximal portion of the inner frame flares radially outward in a direction from the distal portion of the inner frame towards the proximal portion of the inner frame.

Example 59: The prosthetic valve of any example herein, in particular' Examples 46-58, wherein the sealing body includes a frame having a plurality of struts with distal tips of the plurality of struts flared radially outward.

Example 60: The prosthetic valve of any example herein, in particular Examples 46-59, wherein the plurality of anchors are adapted to press one or more native valve leaflets against the sealing body.

Example 61: The prosthetic valve of any example herein, in particular Examples 46-60, wherein the plurality of anchors are adapted to hook around a distal end of a native leaflet of the native valve and retain the native leaflet between the plurality of anchors and the sealing body.

Example 62: The prosthetic valve of any example herein, in particular Examples 46-51, wherein the one or more grip features are adapted to reduce movement of one or more native valve leaflets relative to the sealing body.

Example 63: The prosthetic valve of any example herein, in particular Examples 46-62, wherein the plurality of anchors are adapted to press one or more native valve leaflets towards the one or more grip features to resist a distal movement of the prosthetic valve.

Example 64: The prosthetic valve of any example herein, in particular Examples 46-63, wherein the one or more grip features are adapted to engage a surface of the native valve upon one of the anchors failing to capture one or more native valve leaflets.

Example 65: The prosthetic valve of any example herein, in particular Examples 46-64, wherein the one or more grip features comprise one or more barbs.

Example 66: The prosthetic valve of any example herein, in particular Example 65, wherein the one or more barbs extend distally.

Example 67: The prosthetic valve of any example herein, in particular Example 65 or Example 66, wherein the sealing body includes a frame having a plurality of struts and the one or more barbs are positioned on at least one of the plurality of struts.

Example 68: The prosthetic valve of any example herein, in particular Example 67, wherein the plurality of struts bound a plurality of openings of the frame of the sealing body, and the one or more barbs extend towards at least one of the plurality of openings.

Example 69: The prosthetic valve of any example herein, in particular Examples 65-68, wherein the one or more barbs are positioned for the plurality of anchors to press a native valve leaflet against the one or more barbs.

Example 70: The prosthetic valve of any example herein, in particular Examples 46-69, wherein the prosthetic valve is for deployment to a mitral valve or a tricuspid valve.

Example 71: A method comprising: deploying a prosthetic valve to a native valve, the prosthetic valve including: a plurality of prosthetic valve leaflets, an inner frame supporting the plurality of prosthetic valve leaflets and having a proximal portion and a distal portion, a plurality of anchors positioned at the distal portion of the inner frame and adapted to couple to the native valve, a sealing body positioned radially outward from the inner frame and having a proximal end and a distal end that is coupled to the distal portion of the inner frame such that the sealing body is adapted to move with respect to the inner frame, and one or more grip features positioned on the sealing body and adapted to engage a portion of the native valve.

Example 72: The method of any example herein, in particular Example 71, wherein one or more flexible bodies couple the distal end of the sealing body to the distal portion of the inner frame and allow the sealing body to move with respect to the inner frame.

Example 73: The method of any example herein, in particular Example 71 or Example 72, wherein the inner frame extends about a first axis and the sealing body extends about a second axis, and the distal end is coupled to the distal portion of the inner frame such that the sealing body is adapted to tilt with respect to the inner frame to offset the first axis from the second axis.

Example 74: The method of any example herein, in particular Examples 71-73, wherein the distal end is coupled to the distal portion of the inner frame such that the sealing body is adapted to move axially with respect to the inner frame.

Example 75: The method of any example herein, in particular Examples 71-74, wherein the sealing body is spaced from the inner frame with an annular gap and the proximal end of the sealing body forms an opening for the annular gap.

Example 76: The method of any example herein, in particular Examples 71-75, wherein the sealing body is spaced from the inner frame with an annular gap and the proximal end of the sealing body forms an opening for the annular gap.

Example 77: The method of any example herein, in particular Examples 71-76, wherein the scaling body is spaced from the inner frame with an annular' gap and a fabric bridge extends from the proximal end of the sealing body to the proximal portion of the inner frame to close a proximal end portion of the annular gap.

Example 78: The method of any example herein, in particular Examples 71-77, wherein the inner frame comprises a first frame and the sealing body includes a second frame and a sealing skirt.

Example 79: The method of any example herein, in particular Examples 71-78, wherein the sealing body is tapered radially inward in a direction from the proximal end towards the distal end.

Example 80: The method of any example herein, in particular Examples 71-79, wherein the native valve is a mitral heart valve or a tricuspid heart valve.

Example 81: A prosthetic valve for deployment to a native valve, the prosthetic valve comprising: a valve body; and a plurality of prosthetic valve leaflets coupled to the valve body, each of the plurality of prosthetic valve leaflets including: an outflow edge, a first side commissure end, a second side commissure end positioned opposite the first side commissure end, a central portion positioned between the first side commissure end and the second side commissure end, and an inflow edge positioned opposite the outflow edge, the inflow edge including at least one indentation towards the central portion.

Example 82: The prosthetic valve of any example herein, in particular Example 81, wherein the first side commissure end and the second side commissure end both extend axially.

Example 83: The prosthetic valve of any example herein, in particular Example 81 or Example 82, wherein the at least one indentation is concave with respect to the central portion.

Example 84: The prosthetic valve of any example herein, in particular' Examples 81-83, wherein the inflow edge includes an outer portion, an inner portion, and a mid portion between the outer portion and the inner portion, and the outer portion is convex with respect to the central portion, the mid portion comprises at least one indentation, and the inner portion is convex with respect to the central portion.

Example 85: The prosthetic valve of any example herein, in particular Examples 81-84, wherein each of the plurality of prosthetic valve leaflets includes at least two indentations.

Example 86: The prosthetic valve of any example herein, in particular' Examples 81-85, wherein each of the plurality of prosthetic valve leaflets is symmetrical about an axially extending mid-line of the respective prosthetic valve leaflet.

Example 87: The prosthetic valve of any example herein, in particular Examples 81-86, wherein the at least one indentation is positioned at least 40% of the distance along the respective prosthetic valve leaflet from the outflow edge towards the inflow edge.

Example 88: The prosthetic valve of any example herein, in particular Examples 81-87, wherein the inflow edge comprises a coupling line.

Example 89: The prosthetic valve of any example herein, in particular' Examples 81-88, wherein the inflow edge comprises a suture line.

Example 90: The prosthetic valve of any example herein, in particular Examples 81-89, wherein the plurality of prosthetic valve leaflets comprises at least three prosthetic valve leaflets.

Example 91: The prosthetic valve of any example herein, in particular Examples 81-90, wherein the plurality of prosthetic valve leaflets reduces a size of a neo-sinus space.

Example 92: The prosthetic valve of any example herein, in particular Examples 81-91, wherein the outflow edges of the plurality of prosthetic valve leaflets comprise coaptation surfaces.

Example 93: The prosthetic valve of any example herein, in particular Examples 81-92, wherein the first side commissure end and the second side commissure end each comprise a tab.

Example 94: The prosthetic valve of any example herein, in particular' Examples 81-93, wherein the valve body includes a frame supporting the plurality of prosthetic valve leaflets.

Example 95: The prosthetic valve of any example herein, in particular Examples 81-94, wherein the prosthetic valve is for deployment to a mitral valve or a tricuspid valve.

Example 96: A method comprising: deploying a prosthetic valve to a native valve, the prosthetic valve including: a valve body, and a plurality of prosthetic valve leaflets coupled to the valve body, each of the plurality of prosthetic valve leaflets including: an outflow edge, a first side commissure end, a second side commissure end positioned opposite the first side commissure end, a central portion positioned between the first side commissure end and the second side commissure end, and an inflow edge positioned opposite the outflow edge, the inflow edge including at least one indentation towards the central portion.

Example 97: The method of any example herein, in particular Example 96, wherein the first side commissure end and the second side commissure end both extend axially.

Example 98: The method of any example herein, in particular Example 96 or Example 97, wherein the at least one indentation is concave with respect to the central portion.

Example 99: The method of any example herein, in particular Examples 96-98, wherein the inflow edge includes an outer portion, an inner portion, and a mid portion between the outer portion and the inner portion, and the outer portion is convex with respect to the central portion, the mid portion comprises at least one indentation, and the inner portion is convex with respect to the central portion.

Example 100: The method of any example herein, in particular Examples 96-99, wherein each of the plurality of prosthetic valve leaflets includes at least two indentations.

Example 101: The method of any example herein, in particular Examples 96-100, wherein each of the plurality of prosthetic valve leaflets is symmetrical about an axially extending mid-line of the respective prosthetic valve leaflet.

Example 102: The method of any example herein, in particular Examples 96-101, wherein the at least one indentation is positioned at least 40% of the distance along the respective prosthetic valve leaflet from the outflow edge towards the inflow edge.

Example 103: The method of any example herein, in particular Examples 96-102, wherein the inflow edge comprises a coupling line.

Example 104: The method of any example herein, in particular Examples 96-103, wherein the plurality of prosthetic valve leaflets reduces a size of a neo-sinus space.

Example 105: The method of any example herein, in particular Examples 96-104, wherein the native valve is a mitral heart valve or a tricuspid heart valve.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member can be referred to and claimed individually or in any combination with other group members disclosed herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that can vary, yet is capable of performing the desired operation or process discussed herein.

The terms “a,” “an,” “the” and similar referents used in the context of describing the systems, apparatuses, and methods (especially in the context of the following claims) are to be construed to cover both the singular' and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An apparatus comprising a prosthetic valve for deployment in a native atrioventricular valve, the prosthetic valve comprising:
three prosthetic valve leaflets positioned about a flow channel of the prosthetic valve, each of the prosthetic valve leaflets including:
two commissure ends, and
a central portion positioned between the commissure ends;
wherein the three leaflets are attached to form three commissure regions at which one of the commissure ends of one of the leaflets meets one of the commissure ends of another one of the leaflets;
a valve frame to which the three commissure regions are affixed for supporting the prosthetic valve leaflets, wherein the valve frame provides three openings between the three commissure regions; and
an outer sealing frame having an upstream portion and a downstream portion, the sealing frame being coupled to the valve frame such that, once the prosthetic valve has been deployed in the native atrioventricular valve:
the downstream portion is spaced radially outward from the valve frame to engage and seal against the native atrioventricular valve while defining a gap between the downstream portion and the valve frame, and
ventricular blood passes along radially outward facing surfaces of the prosthetic valve leaflets and through the openings into the gap in a manner that washes out the downstream portion to reduce thrombus formation.

2. The apparatus according to claim 1, wherein the sealing frame has a concave shape facing a ventricle for funneling blood toward the valve frame and the prosthetic valve leaflets.

3. The apparatus according to claim 1, wherein the prosthetic valve further comprises a plurality of curved elongate anchors extending from the valve frame and adapted to capture native leaflets of the native atrioventricular valve.

4. The apparatus according to claim 3, wherein the curved elongate anchors are configured to bend during deployment.

5. The apparatus according to claim 1, wherein the sealing frame has one or more radially-facing grip features adapted to grip the native atrioventricular valve.

6. The apparatus according to claim 1, wherein the sealing frame has an hourglass shape.

7. The apparatus according to claim 1, wherein the valve frame comprises three cantilevered support arms extending in an axial direction, each of the commissure regions being affixed to a respective one of the cantilevered support arms.

8. The apparatus according to claim 7, wherein the openings are between the cantilevered support arms and are substantially unobstructed.

9. The apparatus according to claim 7, wherein the three cantilevered support arms are flexible in a radial direction.

10. The apparatus according to claim 7, wherein each cantilevered support arm further comprises an outer curved strut for resisting inward bending of a respective one of the cantilevered support arms.

11. The apparatus according to claim 10, wherein each outer curved strut is circumferentially aligned with a respective one of the commissure regions.

12. The apparatus according to claim 1, further comprising a fabric sealing skirt positioned over a substantial portion of the sealing frame.

13. The apparatus according to claim 1, wherein an inflow portion of the valve frame is coupled to an inflow portion of the sealing frame.

14. The apparatus according to claim 1, wherein an outlet end of the valve frame is positioned further downstream than an outlet end of the sealing frame.

15. The apparatus according to claim 1, wherein an outlet end of the valve frame does not contact an outlet end of the sealing frame and wherein a clearance is provided therebetween.

16. The apparatus according to claim 1, wherein the prosthetic valve further comprises three flow barriers, each of which extends between a respective one of the commissure regions and the sealing frame, thereby dividing the gap into three discrete regions arranged around the valve frame.

17. The apparatus according to claim 16, wherein each of the flow barriers has a wedge shape.

18. The apparatus according to claim 16, wherein each of the flow barriers has a tapered shape.

19. The apparatus according to claim 1, further comprising an anchoring frame positioned along an inlet end of the prosthetic valve.

20. The apparatus according to claim 1, wherein the sealing frame is formed by a strut lattice.
